SEQUENCE LISTING (54 sequences)

SEQ ID NO: 1 - DNA, 265 bp, severe acute respiratory syndrome coronavirus 2 (SARS-CoV-2)
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 2 - RNA, 265 nt, severe acute respiratory syndrome coronavirus 2
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaag

SEQ ID NO: 3 - DNA, 229 bp, severe acute respiratory syndrome coronavirus 2
caatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 4 - RNA, 229 nt, severe acute respiratory syndrome coronavirus 2
caaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 5 - DNA, 540 bp, severe acute respiratory syndrome coronavirus 2
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggg

SEQ ID NO: 6 - RNA, 540 nt, severe acute respiratory syndrome coronavirus 2
auggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggg

SEQ ID NO: 7 - protein, 180 aa, severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 8 - DNA, 117 bp, severe acute respiratory syndrome coronavirus 2
atgggctatataaacgttttcgcttttccgtttacgatatatagtctactcttgtgcagaatgaattctcgtaactacatagcacaagtagatgtagttaactttaatctcacatag

SEQ ID NO: 9 - RNA, 117 nt, severe acute respiratory syndrome coronavirus 2
augggcuauauaaacguuuucgcuuuuccguuuacgauauauagucuacucuugugcagaaugaauucucguaacuacauagcacaaguagauguaguuaacuuuaaucucacauag

SEQ ID NO: 10 - protein, 38 aa, severe acute respiratory syndrome coronavirus 2
MGYINVFAFPFTIYSLLLCRMNSRNYIAQVDVVNFNLT

SEQ ID NO: 11 - DNA, 1038 bp, severe acute respiratory syndrome coronavirus 2
agtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaa

SEQ ID NO: 12 - RNA, 1038 nt, severe acute respiratory syndrome coronavirus 2
aguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaa

SEQ ID NO: 13 - protein, 346 aa, severe acute respiratory syndrome coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 14 - DNA, 1260 bp, severe acute respiratory syndrome coronavirus 2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 15 - RNA, 1260 nt, severe acute respiratory syndrome coronavirus 2
augucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaa

SEQ ID NO: 16 - protein, 419 aa, severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 17 - DNA, 828 bp, severe acute respiratory syndrome coronavirus 2
atggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaa

SEQ ID NO: 18 - RNA, 828 nt, severe acute respiratory syndrome coronavirus 2
auggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaa

SEQ ID NO: 19 - protein, 275 aa, severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 20 - DNA, 228 bp, severe acute respiratory syndrome coronavirus 2
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 21 - RNA, 228 nt, severe acute respiratory syndrome coronavirus 2
auguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaa

SEQ ID NO: 22 - protein, 75 aa, severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 23 - DNA, 1080 bp, severe acute respiratory syndrome coronavirus 2
atggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 24 - RNA, 1080 nt, severe acute respiratory syndrome coronavirus 2
auggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaa

SEQ ID NO: 25 - DNA, 24 bp, severe acute respiratory syndrome coronavirus 2
actcatgcagaccacacaaggcag

SEQ ID NO: 26 - RNA, 24 nt, severe acute respiratory syndrome coronavirus 2
acucaugcagaccacacaaggcag

SEQ ID NO: 27 - DNA, 800 bp, Artificial Sequence
  Synthetic defective interfering coronaviridae
cttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttcgaacagccctatgtgtcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 28 - RNA, 800 nt, Artificial Sequence
  Synthetic defective interfering coronaviridae
cuuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuucgaacagcccuaugugucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 29 - DNA, 1061 bp, Artificial Sequence
  Synthetic defective interfering coronaviridae
cttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttcgaacagccctatgtgcgaaggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 30 - RNA, 1061 nt, Artificial Sequence
  Synthetic defective interfering coronaviridae
cuuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuucgaacagcccuaugugcgaaggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 31 - DNA, 2522 bp, Artificial Sequence
  Synthetic defective interfering coronaviridae
cttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttcgaacagccctatgtgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctatcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 32 - RNA, 2522 nt, Artificial Sequence
  Synthetic defective interfering coronaviridae
cuuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuucgaacagcccuaugugacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 33 - DNA, 2808 bp, Artificial Sequence
  Synthetic defective interfering coronaviridae
cttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttcgaacagccctatgtgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttcgaaggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaacctttggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagtcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 34 - RNA, 2808 nt, Artificial Sequence
  Synthetic defective interfering coronaviridae
cuuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuucgaacagcccuaugugacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuucgaaggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuuuggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 35 - DNA, 3143 bp, Artificial Sequence
  Synthetic defective interfering coronaviridae
cttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttcgaacagccctatgtgtcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttcgaaggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaacctttggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 36 - RNA, 3143 nt, Artificial Sequence
  Synthetic defective interfering coronaviridae
cuuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuucgaacagcccuaugugucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuucgaaggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuuuggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 37 - DNA, 22 bp, Artificial Sequence
  Primer 1
cgcctacactaattcgtcctga

SEQ ID NO: 38 - DNA, 21 bp, Artificial Sequence
  Primer 2
tgagtcatggctgtgattacg

SEQ ID NO: 39 - DNA, 22 bp, Artificial Sequence
  Primer 3
gacttaaaacactgagccgtac

SEQ ID NO: 40 - DNA, 23 bp, severe acute respiratory syndrome coronavirus 2
gcacaagctgatgagtacgaact

SEQ ID NO: 41 - RNA, 24 nt, severe acute respiratory syndrome coronavirus 2
gcacaagcugaugaguacgaacuu

SEQ ID NO: 42 - DNA, 19 bp, Artificial Sequence
  T7 Promoter
taatacgactcactatagg

SEQ ID NO: 43 - DNA, 21 bp, severe acute respiratory syndrome coronavirus 2
agcttggcactgatccttatg

SEQ ID NO: 44 - DNA, 24 bp, severe acute respiratory syndrome coronavirus 2
acatcaacaccatcaacttttgtg

SEQ ID NO: 45 - DNA, 24 bp, severe acute respiratory syndrome coronavirus 2
ttacccgtgaactcatgcgacagg

SEQ ID NO: 46 - DNA, 20 bp, severe acute respiratory syndrome coronavirus 2
atcagaggcacgtcaacatc

SEQ ID NO: 47 - DNA, 23 bp, severe acute respiratory syndrome coronavirus 2
ttcattctgcacaagagtagact

SEQ ID NO: 48 - DNA, 23 bp, Artificial Sequence
  DI0 Probe
  ref <308>: DATABASE ACCESSION NUMBER: MW250350
  ref <309>: DATABASE ENTRY DATE: 2020-11-29
  ref <313>: RELEVANT RESIDUES IN SEQ ID NO: (1)..(23)
agccctatgtgtcgcttttccgt

SEQ ID NO: 49 - DNA, 21 bp, severe acute respiratory syndrome coronavirus 2
ccctgtgggttttacacttaa

SEQ ID NO: 50 - DNA, 19 bp, severe acute respiratory syndrome coronavirus 2
acgattgtgcatcagctga

SEQ ID NO: 51 - DNA, 27 bp, severe acute respiratory syndrome coronavirus 2
ccgtctgcggtatgtggaaaggttatg

SEQ ID NO: 52 - DNA, 21 bp, Artificial Sequence
  ACTB Primer 7 (F)
aggattcatatgtgggcgatg

SEQ ID NO: 53 - DNA, 21 bp, Artificial Sequence
  ACTB Primer 8 (R)
agctcattgtagaaggtgtgg

SEQ ID NO: 54 - DNA, 21 bp, Artificial Sequence
  ACTB Probe
agcacggcatcgtcaccaact

(19) United States
(12) Patent Application Publication
(10) Pub. No.: US 2023/0285544 A1
(43) Pub. Date: Sep. 14, 2023

(54) SYNTHETIC DEFECTIVE INTERFERING CORONAVIRUSES

(71) Applicant: THE PENN STATE RESEARCH FOUNDATION, University Park, PA (US)

(72) Inventor: Marco ARCHETTI, State College, PA (US)

(21) Appl. No.: 18/018,475

(22) PCT Filed: Aug. 3, 2021
(86) PCT No.: PCT/US2021/044313

§ 371 Date: Jan. 27, 2023

Related U.S. Application Data
(60) Provisional application No. 63/060,327, filed on Aug. 3, 2020.
(60) Provisional application No. 63/116,372, filed on Nov. 20, 2020.

(51) Int. Cl. A61K 39/215; A61P 31/14; C12N 7/00

(52) U.S. Cl. CPC A61K 39/215; A61P 31/14; C12N 7/00; A61K 2039/5258

(57) ABSTRACT

The present disclosure is directed to a recombinant nucleic acid construct encoding a defective interfering coronaviridae virus particle. The recombinant construct comprises: a nucleotide sequence encoding coronaviridae replication signals, wherein said nucleotide sequence of said recombinant nucleic acid construct does not encode one or more functional coronaviridae proteins. Defective interfering coronaviridae virus-like particles produced from the recombinant nucleic acid construct are also disclosed along with methods of treating a subject having a corona virus infection using the virus-like particle.

### CROSS-REFERENCE TO RELATED APPLICATIONS

This application claims priority to U.S. Provisional Application 63/060,327 filed on Aug. 3, 2020 and U.S. Provisional Application 63/116,372 filed on Nov. 20, 2020, each of which is incorporated herein by reference in its entirety.

### FIELD

The present disclosure relates to compositions and methods of treating a viral infection, in particular a human coronavirus.

### BACKGROUND

Technologies to treat and cure diseases causes by viruses belong to two main types: vaccines and antiviral drugs. Vaccines can be made from attenuated viruses that can still replicate; inactivated viruses that cannot replicate; antigenic viral proteins; or virus-like particles with no viral genetic material. In all cases, a vaccine prepares the body to provoke an immune response if exposed to the virus. Antiviral drugs impair viral proteins after an infection has occurred. Antiviral drugs target proteins unique to the virus and essential for their replication cycle. Both types have drawbacks, and for many viruses no effective vaccine or drug is available. Even when drugs are initially effective against a virus, antigenic variation and viral evolution in response to selective pressure from the treatment lead to resistant strains of the virus, which make some therapies ineffective in the long term. A therapy that is effective in the short term and stable against the evolution of resistance in the long term is desirable.

### SUMMARY

A first aspect of the present disclosure is directed to a recombinant nucleic acid construct encoding a defective interfering coronaviridae virus particle. The recombinant construct comprises: a nucleotide sequence encoding coronaviridae replication signals, wherein said nucleotide sequence of said recombinant nucleic acid construct does not encode one or more functional coronaviridae proteins.

Other aspects of the disclosure are directed to a vector comprising the recombinant nucleic acid construct as described herein, and a host cell comprising the vector.

Another aspect of the disclosure is directed to a defective interfering coronaviridae virus particle produced from the recombinant nucleic acid constructs disclosed herein, the vector of comprising the recombinant nucleic acid constructs, or the host cells comprising the vector as described herein.

Another aspect of the disclosure is directed to a pharmaceutical composition comprising the defective interfering coronaviridae virus particle as disclosed herein and a pharmaceutically acceptable carrier.

Another aspect of the present disclosure is directed to a method of treating a subject infected with a coronaviridae virus. This method involves administering to said subject the pharmaceutical composition comprising the defective interfering coronaviridae virus particle as disclosed herein in an amount effective to impair replication and spread of the coronaviridae virus in the patient.

### BRIEF DESCRIPTION OF THE DRAWINGS

FIGS. 1a-1b provide schematics of defective interfering (DI) virus construct. The top half of FIG. 1a shows a map of the full genome of SARS-CoV-2 (NCBI accession NC_045512). Arrows show ORFs; standard symbols for genes are used. The bottom half of FIG. 1a shows two recombinant constructs encoding the expression of two types of synthetic DI coronaviridae viruses in which the parts corresponding to the full-length genome are highlighted. FIG. 1b is a schematic of DI cloning, transcription, transfection, and passage. In order to produce synthetic DI viruses, DNA constructs corresponding to the RNA sequence of DI or DI₀ were transcribed into RNA in vitro using T7 RNA polymerase and transfected into Vero-E6 cells that were then infected with SARS-CoV-2. The supernatant from these cell cultures was used to infect new cells.

FIG. 2 provides a graph showing the fold increase in synthetic recombinant RNA of a coronaviridae DI virus in transfected Vero cells and subsequently infected with SARS-CoV-2.

FIG. 3 provides a graph showing the fold increase in synthetic recombinant RNA of a DI virus 24 hours after the supernatant collected from cell cultures transfected with synthetic recombinant RNA constructs encoding a DI coronaviridae virus and infected with the virus is used to infect new cells.

FIG. 4 is a graph showing a decrease in full-length, wild type (WT) SARS-CoV-2 RNA in cells transfected with the synthetic recombinant RNA constructs encoding a DI virus as described herein and subsequently infected with full-length, wild type SARS-CoV-2.

FIG. 5 provides constructs of WT SARS-CoV-2 and two synthetic constructs used to express DI coronaviridae virus-like particles as disclosed herein.

FIG. 6 is a graphical representation of viral replication in cells infected with WT SARS-CoV-2 and the synthetic DI construct as described in Example 4.

FIGS. 7a-7c are graphs showing growth rates of WT SARs-CoV-2 alone (control) or when coinfected with DI construct (coinfections). Absolute growth amount relative to the amount at 4 hours is shown in FIG. 7a; growth of coinfection relative to controls at the same time point is shown in FIG. 7b; and detail at 24 hours is shown in FIG. 7c.

FIG. 8 provides a graph reporting transmission efficiency of WT SARs-CoV-2 vs WT SARs-CoV-2 with DI virus. Twenty-four hours after infection with WT SARs-CoV-2 alone or in combination with DI virus construct, the supernatant was used to infect new cells. The transmission efficiency is the amount measured by qRT-PCR immediately before passaging divided by the average amount measured almost immediately (4 hours) after passaging

FIGS. 9a-f provide graphs depicting growth rates after infection of WT SARs-CoV-2 vs WT SARs-CoV-2 with DI virus. FIG. 9a shows growth rates (absolute amount relative to the amount at 4 hours) of WT SARs-CoV-2 alone as control vs. WT SARs-CoV-2 when coinfected with DI virus. FIG. 9b shows growth of WT SARs-CoV-2 when coinfected with DI virus relative to controls at the same time point, and FIG. 9c show growth detail at 24 hours. FIG. 9d shows growth rates (absolute amount relative to the amount at 4 hours) of WT SARs-CoV-2 and DI virus when coinfected. FIG. 9e depicts growth of DI virus relative to that of WT SARs-CoV-2 in coinfections at the same time point; and FIG. 9f show growth of WT SARs-CoV-2 and DI virus in detail for at 24 hours.

FIG. 10a-e provides a simulation of the dynamics of DI-WT competition. FIG. 10a is a flow diagram of the model. The number of WT genomes (×WT), DI genomes (xDI) and capsids (×C) increase due to production and decline due to degradation and encapsidation. Production is proportional to the amount of resources of the cell (B), which decreases as a logistic function (with steepness z and inflection to) of time (t); and increases as a linear function (for capsids) or as a logistic function (with steepness s and inflection h) of the number of WT genomes (for WT and DI genomes). DI genomes replicates at a rate R relative to WT genomes. Genomes decay at a rate δG; capsids decay at a rate δC. The rates of encapsidation are κ for WT genomes and ωκ for DI genomes; γ is the number of genomes per capsids; η is the capsid/genome ratio. FIG. 10b provides an example of the results: number of WT and DI genomes and capsids over time. The change in the amount of DI, WT and capsids over time for R=3.3 (the value measures empirically); other parameters: n=10; s=10; h=0.5; δG=0.01; δC=0.001; γ=1; η=0.3; κ=0.03, ω=0.9; B=2, z=−0.03; t0=10; starting from 100 WT and 10 DI genomes. FIG. 10c provides examples of the results: fraction of WT and DI genomes over time. The change in frequency of DI and WT over time for two values of R: R=3.3 (left; the value measured empirically) or R=1.3 (right) assuming no depletion of resources (B=2, z=0); other parameters: n=10; s=10; h=0.5; δG=0.01; δC=0.001; γ=1; η=0.3; κ=0.03, ω=0.9. The initial fraction of DI in this example is 0.1, but the results are independent from the initial fraction. FIG. 10d is a summary of the effect of R and n on the results. The color of each cell shows the stable fraction of DI as a function of the replication advantage (R) of the DI genome and the number of genomes within the range of the viral protein produced by the WT genome (n), for different values of h; assuming no resource depletion (B=2, z=0); other parameters: s=10; δG=0.01; δC=0.001; γ=1; η=0.3; κ=0.03, ω=0.9. For h=0.5 the curves show, for different values of s from 1 to 100, the separation between the combination of parameters for which WT goes extinct (below the curve) or remains at a stable polymorphic equilibrium (above). FIG. 10e is a summary of the effect of other parameters on the results. Combinations of R and n below the curves lead to the extinction of WT. The curves are drawn for different values of γ, η, κ and ω; other parameters now shown because differences are undetectable. In all cases, h=0.5, s=10.

FIGS. 11a-c provide graphs showing the amount of intracellular wild type (WT) SARS-CoV-2 in cells infected with SARS-CoV-2 alone (Control) or in combination with the synthetic recombinant RNA construct encoding a DI virus via polymer nanoparticles (“nanoparticles”) or lipofectamine (“lipofection”) as compared to the control at 12 hours (FIG. 11a), 20 hours (FIG. 11b), and 28 hours (FIG. 11c).

FIGS. 12a-c provide graphs showing the amount of WT SARS-CoV-2 in cell supernatant from cells infected with SARS-CoV-2 alone (Control) or in combination with the synthetic recombinant RNA construct encoding a DI virus via polymer nanoparticles (“nanoparticles”) or lipofectamine (“lipofection”) as compared to the control at 12 hours (FIG. 12a), 20 hours (FIG. 12b), and 28 hours (FIG. 12c).

### DETAILED DESCRIPTION

Viruses thrive by exploiting the cells they infect but must also produce viral proteins to replicate and infect other cells. As a consequence, survival of a virus in vivo is susceptible to exploitation by defective versions of itself—defective in that it does not produce such required proteins. A defective viral genome with deletions in protein-coding genes could still replicate in cells co-infected with full-length viruses, and even replicate faster due to its shorter size, interfering with the replication of the virus. Provided herein are synthetic defective interfering versions of SARS-CoV-2, the virus causing the recent Covid-19 pandemic, designed and prepared by assembling parts of the SARS-CoV-2 viral genome that do not code for any functional protein but enable it to be replicated and packaged. This synthetic defective genome replicates three times faster than SARS-CoV-2 in co-infected cells, and interferes with it, reducing the viral load of a cell by half in 24 hours. The synthetic genome is transmitted as efficiently as the full-length genome, confirming the location of the putative packaging signal of SARS-CoV-2. A version of such synthetic construct could be used as a self-promoting antiviral: by enabling replication of the synthetic genome, the virus promotes its own demise.

Before particular embodiments of the invention are described, general concepts and terminology are defined to provide clarity to the disclosure.

The present disclosure is generally directed to therapeutic compositions for the treatment of viral infection, in particular, coronaviridae infection. The therapeutic compositions of the present disclosure encompass virus-like particles of a synthetic defective interfering (“DI”) coronaviridae virus. Accordingly, a first aspect of the present disclosure is directed to recombinant nucleic acid constructs encoding a DI coronaviridae virus-like particle. The recombinant nucleic acid constructs comprise a nucleotide sequence encoding coronaviridae replication signals where the nucleotide sequence of the recombinant nucleic acid construct does not encode one or more functional coronaviridae proteins. As such, any protein expressed by the transcription and/or translation of the recombinant nucleic acid sequence will not be fully functional as compared to a protein expressed by endogenous full length nucleic acid sequences.

In accordance with this and all aspects of the disclosure, coronaviridae virus (also referred to herein as coronavirus) is referring to any virus belonging to the large family of single-stranded RNA viruses with plus strand orientation. These include both human coronaviridae virus (e.g., SARS-CoV-2, SARS-CoV, MERs-CoV, HCoV-NL63, HCoV-229E, HCoV-OC43, and HCoV-HKU1) and animal coronaviridae viruses (e.g., Feline CoV [serotypes I and II], porcine epidemic diarrhea CoV (PEDV), porcine PRCV, porcine TGEV, Dog CCOC, Rabbit RaCoV, etc.).

The recombinant nucleic acid constructs disclosed herein may be any nucleic acid construct, such as DNA, mRNA, or RNA that encode a DI coronaviridae virus-like particle.

Various nucleic acid sequences, such as DNA and RNA sequences, are disclosed herein, as are protein sequences encoded by the DNA and RNA sequences. Table 2 (herein) comprises each of the specific sequences identified herein, along with a description, and the type of nucleic acid described by the sequence. The skilled artisan appreciates that variants of any of these sequences with conserved amino acid changes are provided as further embodiments. A conservative amino acid substitution can be an amino acid substitution that does not alter the relative charge or size characteristics of the polypeptide in which the amino acid substitution is made. Amino acids are sometimes specified using the standard one letter code: Alanine (A), Serine (S), Threonine (T), Aspartic acid (D), Glutamic acid (E), Asparagine (N), Glutamine (Q), Arginine (R), Lysine (K), Isoleucine (I), Leucine (L), Methionine (M), Valine (V), Phenylalanine (F), Tyrosine (Y), Tryptophan (W), Proline (P), Glycine (G), Histidine (H), Cysteine (C). “Hydrophobic amino acids” refers to A, L, I, V, P, F, W, and M; “polar amino acids” refers to G, S, T, Y, C, N, and Q; and “charged amino acids” refers to D, E, H, K, and R. Conservative amino acid substitution can also include amino acid substitutions of those amino acids that are not critical for protein activity, or substitution of amino acids with other amino acids having similar properties (for example, acidic, basic, positively or negatively charged, polar or non-polar, hydrophobic, charged, et cetera) such that the substitutions of a critical amino acid does not substantially alter activity. The following six groups each contain amino acids that are conservative amino acid substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). One of skill in the art appreciates that the above-identified substitutions are not the only possible conservative substitutions. For example, in some instances one may regard all charged amino acids as conservative substitutions for each other whether they are positive or negative. In addition, individual substitutions, deletions or additions which alter, add or delete a single amino acid or a small percentage of amino acids in an encoded sequence can also be conservative amino acid substitutions. Therefore, when protein/peptide sequence is disclosed as having e.g., at least 85% sequence identity to a particular identified protein sequence, at least 85% of the amino acids in the disclosed sequence are the same as the particular identified protein sequence.

Conservative nucleotide substitution in a nucleic acid encoding an isolated protein are also contemplated in the present embodiments. Conservative nucleotide substitutions include but are not limited to those that cause a conservative amino acid substitution in the encoded amino acid sequence. In addition, degenerate conservative nucleotide substitutions can be made in a gene sequence by substituting a codon for an amino acid with a different codon for the same amino acid. Therefore, when a nucleotide sequence is disclosed as having e.g., at least 85% sequence identity to a particular identified nucleic acid molecule sequence, at least 85% of the nucleotides of the sequence are the same as those identified in the nucleic acid sequence.

In any embodiment, the recombinant nucleic acid construct disclosed herein may encode a DI alphacorona virus-like particle. In any embodiment, the recombinant nucleic acid construct disclosed herein may encode a DI betacorona virus-like particle. In any embodiment, the recombinant nucleic acid construct disclosed herein may encode a DI deltacorona virus-like particle. In any embodiment, the recombinant nucleic acid construct disclosed herein may encode a DI gammacorona virus-like particle. In any embodiment, the recombinant nucleic acid construct disclosed herein may encode a DI human coronaviridae virus-like particle. In any embodiment, the recombinant nucleic acid construct may encode a DI human betacoronavirus virus-like particle. In any embodiment, the recombinant nucleic acid construct disclosed herein may encode a defective human severe acute respiratory syndrome coronavirus. In any embodiment, a recombinant nucleic acid construct may encode a defective human severe acute respiratory syndrome coronavirus-2 (SARS-CoV-2).

In accordance with all aspects of the present disclosure, the recombinant nucleic acid construct comprises a highly deleted form of the coronaviridae genome. In other words, the recombinant nucleic acid construct does not encode a full-length, complete coronaviridae genome. In some embodiments, the recombinant nucleic acid construct comprises a nucleotide sequence that does not encode one or more functional proteins. For example, the recombinant nucleic acid construct does not encode one or more functional coronaviridae packaging proteins, but may encode one or more functional coronaviridae proteins that are not involved in packaging or are alone insufficient to achieve packaging. In any embodiment, a recombinant nucleic acid construct, as disclosed herein, may comprises a nucleotide sequence that does not encode any functional coronaviridae structural proteins. In any embodiment, a recombinant nucleic acid construct, as disclosed herein, may comprise a nucleotide sequence that does not encode any functional coronaviridae proteins.

The nucleotide sequence of the recombinant nucleic acid constructs described herein comprise coronaviridae virus replication signaling elements from one or both of the coronaviridae 5′ untranslated region (5′ UTR) and the coronaviridae 3′ untranslated region (3′ UTR), where the 3′ UTR nucleotide sequence is positioned 3′ to the 5′ UTR nucleotide sequence.

In some embodiments, the portion of the coronaviridae 5′ UTR incorporated into a recombinant nucleic acid construct, as disclosed herein, comprises at least 100 nucleotides of the 5′UTR, at least 150 nucleotides of the 5′UTR, at least 200 nucleotides of the 5′UTR, at least 250 nucleotides of the 5′UTR, at least 300 nucleotides of the 5′UTR, at least 350 nucleotides of the 5′UTR, at least 400 nucleotides of the 5′UTR, at least 450 nucleotides of the 5′UTR, at least 500 nucleotides of the 5′UTR, at least 550 nucleotides of the 5′UTR, at least 600 nucleotides of the 5′UTR, at least 650 nucleotides of the 5′UTR, at least 700 nucleotides of the 5′UTR, at least 750 nucleotides of the 5′UTR, at least 800 nucleotides of the 5′UTR, or at least 850 nucleotides of the 5′UTR. In some embodiments, the recombinant construct comprises the entire coronaviridae 5′ UTR region.

In some embodiments, the portion of the coronaviridae 3′UTR incorporated into a recombinant nucleic acid construct, as disclosed herein, comprises at least 100 nucleotides of the 3′UTR, at least 150 nucleotides of the 3′UTR, at least 200 nucleotides of the 3′UTR, at least 250 nucleotides of the 3′UTR, at least 300 nucleotides of the 3′UTR, at least 350 nucleotides of the 3′UTR, at least 400 nucleotides of the 3′UTR, at least 450 nucleotides of the 3′UTR, at least 500 nucleotides of the 3′UTR, at least 550 nucleotides of the 3′UTR, at least 600 nucleotides of the 3′UTR, at least 650 nucleotides of the 3′UTR, at least 700 nucleotides of the 3′UTR, at least 750 nucleotides of the 3′UTR, at least 800 nucleotides of the 3′UTR, or at least 850 nucleotides of the 3′UTR. In some embodiments, the recombinant nucleic acid construct comprises the entire coronaviridae 3′ UTR region.

In one embodiment, the compositions and methods disclosed herein are related to the SARS-CoV-2 virus, the infectious agent that causes coronavirus severe acute respiratory syndrome (Covid-19). SARS-CoV is closely related to SARS-CoV-2. In SARS-CoV, replication requires the 5′ UTR and the beginning of the nucleotide sequence encoding nsp1, and the 3′ UTR (see Yang D and Leibowitz J L, “The structure and functions of coronavirus genomic 3′ and 5′ ends,” Virus Res. 206:120-133 (2015), which is hereby incorporated by reference in its entirety). Packaging requires about 575-580 nucleotides towards the middle of the nucleotide sequence encoding nsp15 (see, e.g., Hsieh, et al., J Virol. 2005; 79(22):13848-13855, which is hereby incorporated by reference in its entirety).

Based on the information above, and given that the genome of the SARS-CoV-2 virus has been sequenced, synthetic interfering defective SARS-CoV-2 virus-like particles were designed and constructed on the premise that they would be able to be replicated and packaged into virions by cells co-infected by the natural full-length virus. As shown herein, the synthetic interfering virus replicates 3× faster than the wild type SARS-CoV-2 virus, is transmitted with the same efficiency as SARS-CoV-2, and reduces viral load of SARS-CoV-2 within cells by 50% in 24 hours. Thus, these synthetic SARS-CoV-2 viruses are suitable for use as a therapeutic agent to treat Covid-19 infected patients in that they could be used as a self-promoting antiviral, i.e., by enabling replication of the synthetic genome, the virus promotes its own demise.

An exemplary 5′ UTR DNA nucleotide sequence, at least portion of which is suitable for inclusion in a recombinant DNA construct described herein, is the 5′UTR of SARS-CoV-2 DNA (i.e., nucleotides 1-265 of NCBI Ref Sequence NC_045512.2), represented by SEQ ID NO: 1. A recombinant DNA construct for producing a DI coronaviridae virus-like particle, as described herein, may incorporate at least a portion of the 5′ UTR represented by SEQ ID NO: 1 or alternatively, may incorporate at least a portion of a nucleic acid having at least 60%, at least 65%, at least 70%, at least 75%, at least 80%, at least 85%, at least 90%, at least 95% sequence identity to SEQ ID NO: 1.

Similarly, an exemplary 5′ UTR RNA nucleotide sequence, at least portion of which is suitable for inclusion in a recombinant RNA construct described herein, is the 5′UTR of SARS-CoV-2 RNA, represented by SEQ ID NO: 2. A recombinant RNA construct, as described herein, may incorporate at least a portion of the 5′ UTR represented by SEQ ID NO: 2. Additionally or alternatively, a recombinant RNA construct for producing a DI coronaviridae virus-like particle, as described herein, may incorporate at least a portion of a nucleic acid having at least 60%, at least 65%, at least 70%, at least 75%, at least 80%, at least 85%, at least 90%, at least 95% sequence identity to SEQ ID NO: 2.

An exemplary 3′ UTR DNA nucleotide sequence, at least portion of which is suitable for inclusion in a recombinant construct described herein, is the 3′UTR of SARS-CoV-2 DNA (i.e. nucleotides 29675-29903 of NCBI Ref Sequence NC_045512.2), represented by SEQ ID NO: 3. A recombinant construct, as described herein, may incorporate at least a portion of the 3′ UTR represented by SEQ ID NO: 3, or alternatively, may incorporate at least a portion of a nucleic acid having at least 60%, at least 65%, at least 70%, at least 75%, at least 80%, at least 85%, at least 90%, at least 95% sequence identity to SEQ ID NO: 3.

Similarly, an exemplary 5′ UTR RNA nucleotide sequence, at least portion of which is suitable for inclusion in a recombinant RNA construct described herein, is the 5′UTR of SARS-CoV-2 RNA, represented by SEQ ID NO: 4. A recombinant RNA construct, as described herein, may incorporate at least a portion of the 5′ UTR represented by SEQ ID NO: 4 Additionally or alternatively, a recombinant RNA construct for producing a DI coronaviridae virus-like particle, as described herein, may incorporate at least a portion of a nucleic acid having at least 60%, at least 65%, at least 70%, at least 75%, at least 80%, at least 85%, at least 90%, at least 95% sequence identity to SEQ ID NO: 4.

In some embodiments, a recombinant DNA construct may comprise at least a portion of each of the coronaviridae 5′ and 3′ UTR DNA (or nucleotide sequences having at least 60%-95% identity thereto), as described above but no additional coronaviridae elements. In some embodiments, a recombinant RNA construct may comprise at least a portion of each of the coronaviridae 5′ and 3′ UTR RNA (or nucleotide sequences having at least 60%-95% identity thereto), as described above but no additional coronaviridae elements.

Coronaviruses share a common genomic structure that consists of at least six open reading frames (ORFs). The first ORF, ORF1a/b, accounts for about two-thirds of the whole genome length and encodes 16 non-structural proteins (nsps). The others ORFs comprising the remaining one-third of the genome (near the 3′ end) encode at least four main structural proteins: spike (S), membrane (M), envelope (E), and nucleocapsid (N) proteins. See, e.g., Chen, et al., J. Med. Virol. 2020; 92:418-423, which is hereby incorporated by reference in its entirety. Nsp1 is a non-structural protein encoded by ORF1a/b that is involved in cellular mRNA degradation and inhibiting IFN signaling. See, e.g., Chen, et al., J. Med. Virol. 2020; 92:418-423, which is hereby incorporated by reference in its entirety.

As such, in other embodiments, a recombinant DNA construct comprising at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) may further comprise one or more non-functional coronaviridae elements. For example, a recombinant DNA construct may comprise a nucleic acid sequence encoding at least a portion of the coronaviridae virus non-structural protein 1 (nsp1) positioned 3′ to the 5′ UTR nucleotide sequence of the recombinant DNA construct. In some embodiments, the nucleic acid sequence encoding at least a portion of a coronaviridae virus nsp1 incorporated into a recombinant DNA construct comprises less than 100 nucleotides (i.e., will express less than 33 amino acids of nsp1), 101 to 200 nucleotides (i.e., will express 33 to 66 amino acids of nsp1), 201 to 300 nucleotides (i.e., will express 67 to 100 amino acids of nsp1), 301 to 400 nucleotides (i.e., will express 100 to 133 amino acids of nsp1), 401 to 500 nucleotides (i.e., will express 133 to 166 amino acids of nsp1), or 501 to 539 nucleotides (i.e., will express 167 to 179 amino acids of nsp1). In some embodiments, the nucleic acid that ins incorporated into the recombinant DNA sequence expresses full length nsp1.

Similarly, a recombinant RNA construct comprising at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) may further comprise one or more non-functional coronaviridae elements. For example, a recombinant RNA construct may comprise a nucleic acid sequence encoding at least a portion of the coronaviridae virus non-structural protein 1 (nsp1) positioned 3′ to the 5′ UTR nucleotide sequence of the recombinant RNA construct. In some embodiments, the nucleic acid sequence encoding at least a portion of a coronaviridae virus nsp1 incorporated into a recombinant RNA construct comprises less than 100 nucleotides (i.e., will express less than 33 amino acids of nsp1), 101 to 200 nucleotides (i.e., will express 33 to 66 amino acids of nsp1), 201 to 300 nucleotides (i.e., will express 67 to 100 amino acids of nsp1), 301 to 400 nucleotides (i.e., will express 100 to 133 amino acids of nsp1), 401 to 500 nucleotides (i.e., will express 133 to 166 amino acids of nsp1), or 501 to 539 nucleotides (i.e., will express 167 to 179 amino acids of nsp1). In some embodiments, the nucleic acid sequence of nsp1 that is incorporated into the recombinant RNA sequence expresses full length nsp1.

One example of a nucleic acid sequence encoding an nsp1, at least portion of which is suitable for inclusion in a recombinant DNA construct for producing a DI coronaviridae virus-like particle, as described herein, is the nucleic acid sequence that encodes nsp1 from SARS-CoV-2. That is, a recombinant DNA construct as described herein may comprise nucleotides 266-805 of NCBI Ref Sequence NC_045512.2, which is represented by SEQ ID NO: 5, which encodes nsp1 from SARS-CoV-2 (accession number YP_009742608, represented by SEQ ID NO: 7). In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 5 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 7 or a fragment thereof.

A recombinant RNA construct for producing a DI coronaviridae virus-like particle, as described herein, may comprise all or a portion of SEQ ID NO: 6, which encodes nsp1 from SARS-CoV-2 (accession number YP_009742608, represented by SEQ ID NO: 7). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 6 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 7 or a fragment thereof.

In another embodiment, a recombinant DNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) may further comprise a nucleic sequence encoding at least a portion of the coronaviridae ORF10 positioned 5′ to the 3′ UTR nucleotide sequence of the recombinant DNA construct. In some embodiments, the nucleic acid sequence encoding at least a portion of a coronaviridae virus nsp1 incorporated into a recombinant DNA construct comprises 25 to 50 nucleotides (i.e., will express 8 to 16 amino acids of ORF10), 51 to 75 nucleotides (i.e., will express 17-25 amino acids of ORF10), 76 to 100 nucleotides (i.e., will express 25-33 amino acids of ORF10), or 101-114 nucleotides (i.e., will express 33-38 amino acids of ORF10). In some embodiments, the nucleic acid that is incorporated into the recombinant DNA sequence expresses full length ORF10.

Similarly, a recombinant RNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) may further comprise a nucleic sequence encoding at least a portion of the coronaviridae ORF10 positioned 5′ to the 3′ UTR nucleotide sequence of the recombinant RNA construct. In some embodiments, the nucleic acid sequence encoding at least a portion of a coronaviridae virus ORF10 incorporated into a recombinant RNA construct comprises 25 to 50 nucleotides (i.e., will express 8 to 16 amino acids of ORF10), 51 to 75 nucleotides (i.e., will express 17-25 amino acids of ORF10), 76 to 100 nucleotides (i.e., will express 25-33 amino acids of ORF10), or 101-114 nucleotides (i.e., will express 33-38 amino acids of ORF10). In some embodiments, the nucleic acid sequence of ORF10 that is incorporated into the recombinant RNA sequence expresses full length ORF10.

One example of a nucleic acid sequence encoding ORF10, at least portion of which is suitable for inclusion in a recombinant DNA construct for producing a DI coronaviridae virus-like particle, as described herein, is the nucleic acid sequence that encodes ORF1 from SARS-CoV-2 DNA. That is, a recombinant DNA construct as described herein may comprise nucleotides 29558-29674 of NCBI Ref Sequence NC_045512.2, which is represented by SEQ ID NO: 8, which encodes ORF10 from SARS-CoV2 (accession number YP_009725255, represented by SEQ ID NO: 10). In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 8 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 10 or a fragment thereof.

A recombinant RNA construct for producing a DI coronaviridae virus-like particle, as described herein, may comprise all or a portion of SEQ ID NO: 9, which encodes ORF10 from SARS-CoV2 (represented by SEQ ID NO: 10). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 9 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 10 or a fragment thereof.

In another embodiment, a recombinant DNA construct may comprise a nucleic sequence encoding at least a portion of a coronaviridae packaging signaling element positioned 3′ to the 5′ UTR nucleotide sequence of the recombinant DNA construct. In one embodiment, the portion of the coronaviridae packaging signaling element is a portion of the coronaviridae virus non-structural protein 15 (nsp15). Accordingly, a recombinant DNA construct for producing a DI coronaviridae virus-like particle, as described herein, may comprise at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) and further comprises a nucleic sequence encoding at least a portion of the coronaviridae virus non-structural protein 15 (nsp15) positioned 5′ to the 3′ UTR nucleotide sequence of the recombinant DNA construct. In some embodiments, the nucleic acid sequence encoding at least a portion of a coronaviridae virus nsp15 incorporated into a recombinant DNA construct comprises less than 500 nucleotides (i.e., will express less than 166 amino acids of the nsp15 protein), 501 to 600 nucleotides (i.e., will express 167-200 amino acids of the nsp15 protein), 601 to 700 nucleotides (i.e., will express 200-233 amino acids of the nsp15 protein), 700 to 800 nucleotides (i.e., will express 233-266 amino acids of the nsp15 protein), 801 to 900 nucleotides (i.e., will express 267-300 amino acids of the nsp15 protein), or 901-1000 nucleotides (i.e., will express 300-333 amino acids of the nsp15 protein). In some embodiments, the nucleic acid sequence of nsp15 that is incorporated into the recombinant DNA sequence expresses full length nsp15.

Similarly, a recombinant RNA construct may comprise a nucleic sequence encoding at least a portion of a coronaviridae packaging signaling element positioned 3′ to the 5′ UTR nucleotide sequence of the recombinant RNA construct. In one embodiment, the portion of the coronaviridae packaging signaling element is a portion of the coronaviridae nsp15. Thus, a recombinant RNA construct for producing a DI coronaviridae virus-like particle, as described herein, comprises at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) and may further comprise a nucleic sequence encoding at least a portion of the coronaviridae virus non-structural protein 15 (nsp15) positioned 5′ to the 3′ UTR nucleotide sequence of the recombinant RNA construct. In some embodiments, the nucleic acid sequence encoding at least a portion of a coronaviridae virus nsp15 incorporated into a recombinant RNA construct comprises less than 500 nucleotides (i.e., will express less than 166 amino acids of the nsp15 protein), 501 to 600 nucleotides (i.e., will express 167-200 amino acids of the nsp15 protein), 601 to 700 nucleotides (i.e., will express 200-233 amino acids of the nsp15 protein), 700 to 800 nucleotides (i.e., will express 233-266 amino acids of the nsp15 protein), 801 to 900 nucleotides (i.e., will express 267-300 amino acids of the nsp15 protein), or 901-1000 nucleotides (i.e., will express 300-333 amino acids of the nsp15 protein). In some embodiments, the nucleic acid sequence of nsp15 that is incorporated into the recombinant RNA sequence expresses full length nsp15.

One example of a nucleic acid sequence encoding an nsp15, at least portion of which is suitable for inclusion in a recombinant DNA construct for producing a DI coronaviridae virus-like particle, as described herein, is the nucleic acid sequence that encodes nsp15 from SARS-CoV-2 DNA. That is, a recombinant DNA construct as described herein may comprise nucleotides 19621-20658 of NCBI Ref Sequence NC_045512.2, which is represented by SEQ ID NO: 11, which encodes nsp15 from SARS-CoV-2 (accession number YP_009725310.1, represented by SEQ ID NO: 13). In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 11 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 13 or a fragment thereof.

A recombinant RNA construct for producing a DI coronaviridae virus-like particle, as described herein, may comprise all or a portion of SEQ ID NO: 12, which encodes nsp15 from SARS-CoV-2 (accession number YP_009725310.1, represented by SEQ ID NO: 13). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 12 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 13 or a fragment thereof.

In another embodiment, a recombinant DNA construct may comprise a nucleic sequence encoding at least a portion of a coronaviridae packaging signaling element positioned 3′ to the 5′ UTR nucleotide sequence of the recombinant DNA construct. In one embodiment, the portion of the coronaviridae packaging signaling element is a portion of the coronaviridae N Protein. Accordingly, in one embodiment, the recombinant DNA construct for producing a DI coronaviridae virus-like particle, as described herein, comprises at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) and further comprises a nucleic sequence encoding at least a portion of the coronaviridae virus N Protein positioned 5′ to the 3′ UTR nucleotide sequence of the recombinant DNA construct. In some embodiments, the nucleic acid sequence encoding at least a portion of a coronaviridae virus N Protein incorporated into a recombinant DNA construct comprises more than 500 nucleotides (i.e., will express at least 166 amino acids of the N Protein), such as 501 to 600 nucleotides (i.e., will express 167-200 amino acids of N Protein), 601 to 700 nucleotides (i.e., will express 200-233 amino acids of N Protein), 700 to 800 nucleotides (i.e., will express 233-266 amino acids of N Protein), 801 to 900 nucleotides (i.e., will express 267-300 amino acids of N Protein), 901-1000 nucleotides (i.e., will express 300-333 amino acids of N Protein), or 1001 to 1100 nucleotides (i.e., will express 333-366 amino acids of N Protein). In some embodiments, the nucleic acid sequence of N Protein that is incorporated into the recombinant DNA sequence expresses full length N Protein.

Similarly, a recombinant RNA construct may comprise a nucleic sequence encoding at least a portion of a coronaviridae packaging signaling element positioned 3′ to the 5′ UTR nucleotide sequence of the recombinant RNA construct. In one embodiment, the portion of the coronaviridae packaging signaling element is a portion of the coronaviridae N Protein. Thus, a recombinant RNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) and further comprise a nucleic sequence encoding at least a portion of the coronaviridae virus N Protein positioned 5′ to the 3′ UTR nucleotide sequence of the recombinant RNA construct. In some embodiments, the nucleic acid sequence encoding at least a portion of a coronaviridae virus N Protein incorporated into a recombinant RNA construct comprises more than 500 nucleotides (i.e., will express at least 166 amino acids of the N Protein), such as 501 to 600 nucleotides (i.e., will express 167-200 amino acids of N Protein), 601 to 700 nucleotides (i.e., will express 200-233 amino acids of N Protein), 700 to 800 nucleotides (i.e., will express 233-266 amino acids of N Protein), 801 to 900 nucleotides (i.e., will express 267-300 amino acids of N Protein), 901-1000 nucleotides (i.e., will express 300-333 amino acids of N Protein), or 1001 to 1100 nucleotides (i.e., will express 333-366 amino acids of N Protein). In some embodiments, the nucleic acid sequence of the N Protein that is incorporated into the recombinant RNA sequence expresses full length N Protein.

One example of a nucleic acid sequence encoding N Protein, at least portion of which is suitable for inclusion in a recombinant DNA construct for producing a DI coronaviridae virus-like particle, as described herein, is the nucleic acid sequence that encodes N Protein from SARS-CoV-2 DNA. That is, a recombinant DNA construct as described herein may comprise nucleotides 28274-29533 of NCBI Ref. Sequence NC_045512.2, which is represented by SEQ ID NO: 14, which encodes N Protein from SARS-CoV-2 (accession number YP_009724397.2, represented by SEQ ID NO: 16). In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 14 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 16 or a fragment thereof.

A recombinant RNA construct for producing a DI coronaviridae virus-like particle, as described herein, may comprise the nucleic acid sequence that encodes N Protein from SARS-CoV-2 DNA. That is, a recombinant RNA construct as described herein may comprise all or a portion of SEQ ID NO: 15, which encodes N Protein from SARS-CoV-2 (accession number YP_009724397.2, represented by SEQ ID NO: 16). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 15 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 16 or a fragment thereof.

In another embodiment, a recombinant DNA construct as described herein may comprise a nucleic sequence encoding ORF3a, where the nucleic acid sequence encoding ORF3a is positioned 5′ to the 3′ UTR nucleotide sequence of the recombinant DNA construct. For example, a recombinant DNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) may further comprise a nucleic sequence encoding at least a portion of the coronaviridae virus ORF3a protein positioned 5′ to the 3′ UTR nucleotide sequence of the recombinant DNA construct. In some embodiments, the nucleic acid sequence encoding at least a portion of a coronaviridae virus ORF3a incorporated into a recombinant DNA construct comprises less than 100 nucleotides (i.e., will express less than 33 amino acids of ORF3a), 101 to 200 nucleotides (i.e., will express 33-66 amino acids of ORF3a), 201 to 300 nucleotides (i.e., will express 67-100 amino acids of ORF3a), 301 to 400 nucleotides (i.e., will express 100-133 amino acids of ORF3a), 401 to 500 nucleotides (i.e., will express 133-166 amino acids of ORF3a), or 501-600 nucleotides (i.e., will express 167-200 amino acids of ORF3a). In some embodiments, the nucleic acid sequence of ORF3a that is incorporated into the recombinant DNA sequence expresses full length ORF3a.

Similarly, a recombinant RNA construct may comprise a nucleic sequence encoding at least a portion of a ORF3a positioned 5′ to the 3′ UTR nucleotide sequence of the recombinant RNA construct. For example, a recombinant RNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) may further comprise a nucleic sequence encoding at least a portion of the coronaviridae virus ORF3a protein positioned 5′ to the 3′ UTR nucleotide sequence of the recombinant RNA construct. In some embodiments, the nucleic acid sequence encoding at least a portion of a coronaviridae virus ORF3a incorporated into a recombinant RNA construct comprises less than 100 nucleotides (i.e., will express less than 33 amino acids of ORF3a), 101 to 200 nucleotides (i.e., will express 33-66 amino acids of ORF3a), 201 to 300 nucleotides (i.e., will express 67-100 amino acids of ORF3a), 301 to 400 nucleotides (i.e., will express 100-133 amino acids of ORF3a), 401 to 500 nucleotides (i.e., will express 133-166 amino acids of ORF3a), or 501-600 nucleotides (i.e., will express 167-200 amino acids of ORF3a). In some embodiments, the nucleic acid sequence of ORF3a that is incorporated into the recombinant RNA sequence expresses full length ORF3a.

One example of a nucleic acid sequence encoding ORF3a, at least portion of which is suitable for inclusion in a recombinant DNA construct for producing a DI coronaviridae virus-like particle, as described herein, is the nucleic acid sequence that encodes ORF3a from SARS-CoV-2 DNA. That is, a recombinant DNA construct as described herein may comprise nucleotides 25393-26220 of NCBI Ref. Sequence NC_045512.2, which is represented by SEQ ID NO: 17, which encodes ORF3a from SARS-CoV-2 (accession number YP_009724391.1, represented by SEQ ID NO: 19). In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 17 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 19 or a fragment thereof.

A recombinant RNA construct for producing a DI coronaviridae virus-like particle, as described herein, may comprise all or a portion of SEQ ID NO: 18, which encodes ORF3a from SARS-CoV-2 (accession number YP_009724391.1, represented by SEQ ID NO: 19). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 18 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 19 or a fragment thereof.

In another embodiment, a recombinant DNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 1 and/or SEQ ID NO: 3) may further comprise a nucleic sequence encoding at least a portion of the coronaviridae E Protein positioned 5′ to the 3′ UTR nucleotide sequence of the recombinant DNA construct. In some embodiments, the nucleic acid sequence encoding at least a portion of a coronaviridae virus E Protein incorporated into a recombinant DNA construct comprises greater than 50 nucleotides (i.e., will express at least 16 amino acids of E Protein), such as 50 to 100 nucleotides (i.e., will express 16-33 amino acids of E Protein), 101 to 150 nucleotides (i.e., will express 33-50 amino acids of E Protein), or 151 to 200 nucleotides (i.e., will express 50-66 amino acids of E Protein). In some embodiments, the nucleic acid sequence of E Protein that is incorporated into the recombinant DNA sequence expresses full length E Protein.

Similarly, a recombinant RNA construct for producing a DI coronaviridae virus-like particle, as described herein, comprising at least a portion of the coronaviridae 5′ UTR and at least a portion of the coronaviridae 3′ UTR nucleotide sequence of the coronaviridae virus (e.g., SEQ ID NO: 2 and/or SEQ ID NO: 4) may further comprise a nucleic sequence encoding at least a portion of the coronaviridae E Protein positioned 5′ to the 3′ UTR nucleotide sequence of the recombinant RNA construct. In some embodiments, the nucleic acid sequence encoding at least a portion of a coronaviridae virus E Protein incorporated into a recombinant RNA construct comprises greater than 50 nucleotides (i.e., will express at least 16 amino acids of E Protein), such as 50 to 100 nucleotides (i.e., will express 16-33 amino acids of E Protein), 101 to 150 nucleotides (i.e., will express 33-50 amino acids of E Protein), or 151 to 200 nucleotides (i.e., will express 50-66 amino acids of E Protein). In some embodiments, the nucleic acid sequence of E Protein that is incorporated into the recombinant RNA sequence expresses full length E Protein.

One example of a nucleic acid sequence encoding E Protein, at least portion of which is suitable for inclusion in a recombinant DNA construct for producing a DI coronaviridae virus-like particle, as described herein, is the nucleic acid sequence that encodes E Protein from SARS-CoV-2 DNA. That is, a recombinant DNA construct as described herein may comprise nucleotides 26245-26472 of NCBI Ref. Sequence NC_045512.2, which is represented by SEQ ID NO: 20, which encodes E Protein from SARS-CoV-2 (accession number YP_009724392.1, represented by SEQ ID NO: 22. In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 20 or a fragment thereof. Additionally, or alternatively, a recombinant DNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 22 or a fragment thereof.

A recombinant RNA construct for producing a DI coronaviridae virus-like particle, as described herein, may comprise all or a portion of SEQ ID NO: 21, which encodes E Protein from SARS-CoV-2 (accession number YP_009724392.1, represented by SEQ ID NO: 22). In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to SEQ ID NO: 21 or a fragment thereof. Additionally, or alternatively, a recombinant RNA construct for producing a DI coronaviridae virus-like particle, as described herein, comprises a nucleotide sequence encoding a peptide having at least 60%, at least 65%, at least 70%, at least 75%, at least 80%, at least 85%, at least 90%, at least 95% sequence identity to SEQ ID NO: 22 or a fragment thereof.

In some embodiments, the recombinant DNA or RNA construct disclosed herein comprising at least a portion of the coronaviridae 5′ UTR and at least a portion of the coronaviridae 3′ UTR nucleotide sequence of the coronaviridae virus may further comprise a nucleic sequence encoding at least a portion of the coronaviridae ORF3a and E Protein positioned 5′ to the 3′ UTR nucleotide sequence of the recombinant DNA or RNA construct. Suitable DNA and RNA sequences encoding ORF3a and E Protein are disclosed above.

Between the region of the full-length SARS-CoV-2 virus (NCBI Ref. Sequence NC_045512.2) that encodes ORF3a and E Protein, there is a 24 bp nucleotide sequence (SEQ ID NO: 40) which may be included in a recombinant DNA construct, between sequences encoding all or a portion of ORF3a and E protein. Similarly, the corresponding 24 nucleotide sequence (SEQ ID NO. 41) may occur in an RNA construct in the same manner. SEQ ID NO: 23 provides the combined DNA nucleotide sequences that encode ORF3a and E protein, together with the intron there between. Likewise, SEQ ID NO: 24 provides the combined RNA nucleotide sequences that encode ORF3a and E protein, together with the intron there between. In any embodiment, a nucleic acid sequence encoding all or a portion of ORF3a (SEQ ID NOs: 17, 18), all or a portion of E Protein (SEQ ID NO: 20, 21) or all or a portion of a combination thereof (SEQ ID NOs: 23, 24) may be included in a recombinant nucleic acid construct for producing a DI coronaviridae virus-like particle, as described herein.

Recombinant Constructs

A recombinant nucleic acid construct may include at least a portion of any or all of the aforementioned non-structural elements as described above, namely, one or more of at least a portion of the 5′ UTR, 3′ UTR, nsp1-encoding region, nsp15-encoding region, ORF10-endoding region, ORF3a-encoding region, E Protein-encoding region, and N Protein-encoding region of a coronaviridae genomic sequence. In some embodiments, a recombinant nucleic acid construct as described herein further comprises a promoter sequence, such as a T7 promoter, operatively coupled to the 5′ UTR nucleotide sequence of the construct. In some embodiments, a recombinant nucleic acid construct, as described herein, further comprises one or more primer sequences which may or may not have any sequence identity to a coronaviridae genomic sequence.

In any embodiment, a recombinant nucleic acid construct as described herein may be a deoxyribonucleic acid construct. Alternatively, a recombinant nucleic acid construct, as described herein, may be a ribonucleic acid construct, such as RNA or mRNA. The following describe examples of recombinant DNA and RNA-based constructs that may be used but are not intended to be inclusive of all variations and combinations that may be generated and useful within the context of inducing expression of non-functional elements of the coronaviridae genome.

In any embodiment, a recombinant nucleic acid DNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding ORF10, and all or part of a 3′UTR. One non-limiting example of a recombinant nucleic acid construct comprising these components is provided herein as SEQ ID NO: 27, which has a nucleotide sequence that comprises, from 5′→3′, the 5′ UTR of SEQ ID NO: 1 (where n is adenosine (a) or cytosine (c)), nucleotides 1-208 of the nucleotide sequence encoding nsp1 (SEQ ID NO: 5), nucleotides 20-117 of the nucleotide sequence encoding ORF10 (SEQ ID NO: 8), and the 3′UTR of SEQ ID NO: 3. In some embodiments, the nucleotide sequence of a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO:27.

In any embodiment, a recombinant nucleic acid RNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding ORF10, and all or part of a 3′UTR. One non-limiting example of a recombinant RNA construct comprising these components is provided herein as SEQ ID NO: 28, which has a nucleotide sequence that comprises, from 5′→3′, the 5′ UTR of SEQ ID NO: 2 (where n is adenosine (a) or cytosine (c)), nucleotides 1-208 of the nucleotide sequence encoding nsp1 (SEQ ID NO: 6), nucleotides 20-117 of the nucleotide sequence encoding ORF10 (SEQ ID NO: 9), and the 3′UTR of SEQ ID NO: 4. In some embodiments, the nucleotide sequence of a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 28.

In any embodiment, a recombinant nucleic acid DNA construct for producing a DI coronaviridae virus-like particle, as described herein, has a nucleotide sequence that comprises, all or part of a 5′ UTR, all or part of a nucleotide sequence encoding nsp1, all or part of the nucleotide sequence encoding ORF3a+E Protein, all or part of a nucleotide sequence encoding ORF10, and all or part of a 3′UTR. One non-limiting example of a recombinant nucleic acid construct comprising these components is provided herein as SEQ ID NO: 29, which has a nucleotide sequence that comprises, from 5′→3′, the 5′ UTR of SEQ ID NO: 1 (where n is adenosine (a) or cytosine (c)), nucleotides 1-207 of the nucleotide sequence encoding nsp1 (SEQ ID NO: 5), nucleotides 762-1016 of the nucleotide sequence encoding ORF3a+E Protein (SEQ ID NO: 23), nucleotides 20-117 of ORF10 (SEQ ID NO: 8), and the 3′UTR of SEQ ID NO. 3. SEQ ID NO: 29 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, the nucleotide sequence of a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 29 (not including the five optional nucleotides as described above).

In any embodiment, a recombinant nucleic acid RNA construct for producing a DI coronaviridae virus-like particle, as described herein, has a nucleotide sequence that comprises, all or part of a 5′ UTR, all or part of a nucleotide sequence encoding nsp1, all or part of the nucleotide sequence encoding ORF3a+E Protein, all or part of a nucleotide sequence encoding ORF10, and all or part of a 3′UTR. One non-limiting example of a recombinant RNA construct comprising these components is provided herein as SEQ ID NO: 30, which has a nucleotide sequence that comprises, from 5′→3′, the 5′ UTR of SEQ ID NO: 2 (where n is adenosine (a) or cytosine (c)), nucleotides 1-207 of the nucleotide sequence encoding nsp1 (SEQ ID NO: 6), nucleotides 762-1016 of the nucleotide sequence encoding ORF3a+E Protein (SEQ ID NO: 24), nucleotides 20-117 of the nucleotide sequence encoding ORF10 (SEQ ID NO: 9), and the 3′UTR of SEQ ID NO. 4. SEQ ID NO: 30 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, the nucleotide sequence of a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 30 (not including the five optional nucleotides as described above).

In any embodiment, a recombinant nucleic acid DNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding nsp15, all or part of a nucleotide sequence encoding ORF3a+E Protein, all or part of a nucleotide sequence encoding ORF10, and all or part of a 3′UTR. One non-limiting example of a recombinant DNA construct comprising these components is provided herein as SEQ ID NO: 31, which has a nucleotide sequence that comprises, from 5′→3′, a portion of the 5′ UTR of SEQ ID NO: 1 (where n is adenosine (a) or cytosine (c)), nucleotides 1-207 of the nucleotide sequence encoding nsp1 (SEQ ID NO: 5), nucleotides 54-720 of the nucleotide sequence encoding nsp15 (SEQ ID NO: 11), nucleotides 64-2159 of the nucleotide sequence encoding N Protein (SEQ ID NO: 14), nucleotides 20-117 of the nucleotide sequence encoding ORF10 (SEQ ID NO: 8), and the 3′UTR of SEQ ID NO. 3. In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 31.

In any embodiment, a recombinant nucleic acid RNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding nsp15, all or part of a nucleotide sequence encoding ORF3a+E Protein, all or part of a nucleotide sequence encoding ORF10, and all or part of a 3′UTR. One non-limiting example of a recombinant RNA construct comprising these components is provided herein as SEQ ID NO: 32, which has a nucleotide sequence that comprises, from 5′→3′, a portion of the 5′ UTR of SEQ ID NO: 2 (where n is adenosine (a) or cytosine (c)), nucleotides 1-207 of the nucleotide sequence encoding nsp1 (SEQ ID NO: 6), nucleotides 54-720 of the nucleotide sequence encoding nsp15 (SEQ ID NO: 12), nucleotides 205-2159 of the nucleotide sequence encoding N Protein (SEQ ID NO: 15), nucleotides 20-117 of the nucleotide sequence encoding ORF10 (SEQ ID NO: 9), and the 3′UTR of SEQ ID NO. 4. In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 32.

In any embodiment, a recombinant nucleic acid DNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding nsp15, all or part of a nucleotide sequence encoding ORF3a+E Protein, all or part of a nucleotide sequence encoding N Protein, all or part of a nucleotide sequence encoding ORF10, and all or part of a 3′UTR. One non-limiting example of a recombinant nucleic acid construct comprising these components is provided herein as SEQ ID NO: 33, which has a nucleotide sequence that comprises, from 5′→3′, a portion of the 5′ UTR of SEQ ID NO: 1 (where n is adenosine (a) or cytosine (c)), nucleotides 1-207 of the nucleotide sequence encoding nsp1 (SEQ ID NO: 5), nucleotides 54-720 of the nucleotide sequence encoding nsp15 (SEQ ID NO: 11), nucleotides 62-1016 of ORF3a+E protein (SEQ ID NO: 23), nucleotides 205-2159 of the nucleotide sequence encoding N Protein (SEQ ID NO: 14), an intron corresponding to nucleotides 29534-29557 of NCBI Ref. Sequence NC_045512.2 (SEQ ID NO: 25), nucleotides 20-117 of ORF10 (SEQ ID NO: 8), and the 3′UTR of SEQ ID NO: 3. SEQ ID NO: 33 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, a recombinant DNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 33 (not including the five optional nucleotides as described above).

Another non-limiting example of a recombinant nucleic acid construct comprising these components is provided herein as SEQ ID NO: 35, which has a nucleotide sequence that comprises, from 5′→3′, a portion of the 5′ UTR of SEQ ID NO: 1 (where n is adenosine (a) or cytosine (c)), nucleotides 1-524 of the nucleotide sequence encoding nsp1 (SEQ ID NO: 5), nucleotides 54-720 of the nucleotide sequence encoding nsp15 (SEQ ID NO: 11), nucleotides 62-1016 of ORF3a+E protein (SEQ ID NO: 23), nucleotides 205-2159 of the nucleotide sequence encoding N Protein (SEQ ID NO: 14), nucleotides 1-117 of the nucleotide sequence encoding ORF10 (SEQ ID NO: 8), and the 3′UTR of SEQ ID NO: 3. SEQ ID NO: 35 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, a recombinant DNA construct for producing a DI coronaviridae virus-like particle, as described herein, a nucleotide sequence having at least 60%, at least 65%, at least 70%, at least 75%, at least 80%, at least 85%, at least 90%, at least 95% sequence identity to the sequence of SEQ ID NO: 35 (not including the five optional nucleotides as described above).

In any embodiment, a recombinant nucleic acid RNA construct for producing a DI coronaviridae virus-like particle, as described herein, has a nucleotide sequence that comprises, all or part of a 5′ UTR, all or part of a nucleotide sequence encoding nsp1, all or part of a nucleotide sequence encoding nsp15, all or part of a nucleotide sequence encoding ORF3a+E Protein, all or part of a nucleotide sequence encoding N Protein, all or part of a nucleotide sequence encoding ORF10, and all or part of a 3′UTR. An exemplary recombinant RNA construct comprising these components is provided herein as SEQ ID NO: 34, which has a nucleotide sequence that comprises, from 5′→3′, a portion of the 5′ UTR of SEQ ID NO: 2 (where n is adenosine (a) or cytosine (c)), nucleotides 1-207 of the nucleotide sequence encoding nsp1 (SEQ ID NO: 6), nucleotides 54-720 of the nucleotide sequence encoding nsp15 (SEQ ID NO: 12), nucleotides 62-1016 of the nucleotide sequence encoding ORF3a+E protein (SEQ ID NO: 24), nucleotides 205-2159 of the nucleotide sequence encoding N Protein (SEQ ID NO: 15), an intron corresponding to nucleotides 29534-29557 of NCBI Ref. Sequence NC_045512.2 (SEQ ID NO: 26), nucleotides 20-117 of the nucleotide sequence encoding ORF10 (SEQ ID NO: 9), and the 3′UTR of SEQ ID NO: 4. SEQ ID NO: 34 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, a recombinant RNA construct for producing a DI coronaviridae virus-like particle, as described herein, comprises a nucleotide sequence having at least 60%, at least 65%, at least 70%, at least 75%, at least 80%, at least 85%, at least 90%, at least 95% sequence identity to the sequence of SEQ ID NO: 34 (not including the five optional nucleotides as described above).

Another non-limiting example recombinant RNA construct comprising these components is provided herein as SEQ ID NO: 36, which has a nucleotide sequence that comprises, from 5′→3′, a portion of the 5′ UTR of SEQ ID NO: 2 (where n is adenosine (a) or cytosine (c)), nucleotides 1-524 of the nucleotide sequence encoding nsp1 (SEQ ID NO: 6), nucleotides 54-720 of the nucleotide sequence encoding nsp15 (SEQ ID NO: 6), nucleotides 62-1016 of the nucleotide sequence encoding ORF3a+E protein (SEQ ID NO: 24), nucleotides 205-2159 of the nucleotide sequence encoding N Protein (SEQ ID NO: 15), nucleotides 1-117 of the nucleotide sequence encoding ORF10 (SEQ ID NO: 9), and the 3′UTR of SEQ ID NO: 4. SEQ ID NO: 36 also contains 5 nucleotides, underline in Table 2, that are optional and are a “scar” of a restriction site. In any embodiment, an RNA construct may be identical to SEQ ID NO: 36 except for the omission of the underling “cgaa” and “t” nucleotides. In some embodiments, a recombinant RNA construct for producing a DI coronaviridae virus-like particle, as described herein, a nucleotide sequence having at least 60%, at least 65%, at least 70%, at least 75%, at least 80%, at least 85%, at least 90%, at least 95% sequence identity to the sequence of SEQ ID NO: 36 (not including the five optional nucleotides as described above).

In some embodiments, a recombinant nucleic acid construct as described herein further comprise one or more primer/probe nucleotide sequences. In one embodiment, the recombinant nucleic acid construct comprises forward and reverse primer sequences. In some embodiments, the recombinant nucleic acid construct comprises forward and reverse primer sequences along with a probe sequence, e.g., TaqMan primers and probe sequence. An exemplary primer and probe nucleotide sequences comprise the nucleotide sequences of SEQ ID NO: 37, 38, and 39.

Inducing replication and expression of the recombinant nucleic acid construct encoding a DI coronaviridae virus, thereby generating a DI coronaviridae virus-like particle, may be carried out via any expression system known to those skilled in the art. For example, in any aspect, a vector containing the recombinant nucleic acid construct (e.g., RNA, DNA) encoding a DI virus as describe herein (e.g., any of SEQ ID NOs: 27-36) may be used to transfect a host cells with the recombinant nucleic acid construct (which may be in vivo). As used herein, the term “vector” refers to a nucleotide molecule capable of transporting other nucleotides to which it has been linked. One exemplary type of vector is a “plasmid”, which represents a circular double stranded DNA loop into which additional DNA sections can be ligated. Another type of vector is a viral vector wherein additional DNA sections can be ligated with the viral genome. Methods of introducing a DNA into cells are known to those skilled in the art and may include a transformation method, a transfection method, an electroporation method, a nuclear injection method, or a carrier such as a nanoparticle, liposome, micelle, skin cell, or a fusion method using protoplasts.

The present disclosure further discloses and provides a vector comprising a recombinant nucleic acid construct, either DNA or RNA, as described herein, and a host cell comprising this vector.

Suitable host cells can be a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety). In some embodiments, the host cell chosen for expression may be of mammalian origin. Suitable mammalian host cells include, without limitation, Vero cells, COS-1 cells, COS-7 cells, HEK293 cells, BHK21 cells, CHO cells, BSC-1 cells, HeG2 cells, SP2/0 cells, HeLa cells, mammalian myeloma cells, mammalian lymphoma cells, or any derivative, immortalized or transformed cell thereof. Other suitable host cells include, without limitation, yeast cells, insect cells, and plant cells.

The present disclosure further provides a DI coronaviridae virus-like particle produced from any one of the recombinant nucleic acid constructs, the vectors, and host cells as disclosed herein. As noted above, in any embodiment, a DI coronaviridae virus-like particle may be derived from a human coronavirus, for example, and without limitation, the human severe acute respiratory syndrome coronavirus 2 (SARS-CoV-2).

Pharmaceutical Compositions

Another aspect of the present disclosure relates to a pharmaceutical composition that comprises any one of the DI coronaviridae virus-like particles as described herein and a pharmaceutically acceptable carrier.

The pharmaceutical compositions useful herein contain a pharmaceutically acceptable carrier, including any suitable diluent or excipient and a DI coronaviridae virus-like particle generated by any one of the recombinant RNA or DNA constructs as disclosed herein. As used herein, the term “pharmaceutically acceptable” means being approved by a regulatory agency of the Federal or a state government or listed in the U.S. Pharmacopeia, European Pharmacopeia or other generally recognized pharmacopeia for use in vertebrates, and more particularly in humans.

Pharmaceutically acceptable carriers include, but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition), which is incorporated herein by reference for its disclosure of said carriers. The formulation should suit the mode of administration. In a preferred embodiment, the formulation is suitable for administration to humans, preferably is sterile, non-particulate and/or non-pyrogenic.

A composition comprising DI coronaviridae virus-like particles, as disclosed herein, may be supplied as a liquid, as a dry sterilized lyophilized powder or water-free concentrate for reconstitution (e.g., with water or saline to the appropriate concentration) for administration to a subject. These doses may be measured as total number of virus-like particles or as g of any coronaviridae protein described herein. For example, a composition comprising DI coronaviridae virus-like particles may be supplied as a dry sterile lyophilized powder in a hermetically sealed container at a unit dosage of about 1 μg, about 5 μg, about 10 μg, about 20 μg, about 25 μg, about 30 μg, about 50 μg, about 100 μg, about 125 μg, about 150 μg, or about 200 μg. Alternatively, the unit dosage of a composition comprising DI coronaviridae virus-like particles may be less than about 1 ag, (for example about 0.08 ag, about 0.04 μg; about 0.2 μg, about 0.4 μg, about 0.8 μg, about 0.5 μg or less, about 0.25 μg or less, or about 0.1 μg or less), or more than about 125 μg, (for example about 150 μg or more, about 250 μg or more, or about 500 μg or more). A composition comprising DI coronaviridae virus-like particles should be administered within about 12 hours, preferably within about 6 hours, within about 5 hours, within about 3 hours, or within about 1 hour after being reconstituted from a lyophilized powder.

In an alternative embodiment, a composition comprising DI coronaviridae virus-like particles may be supplied in liquid form in a hermetically sealed container indicating the quantity and concentration of a composition comprising DI coronaviridae virus-like particles. Preferably, the liquid form of a composition comprising DI coronaviridae virus-like particles is supplied in a hermetically sealed container at least about 50 μg/mL, more preferably at least about 100 μg/mL, at least about 200 μg/mL, at least 500 μg/mL, or at least 1 mg/mL.

Generally, DI coronaviridae virus-like particles are administered in an effective amount or quantity (as defined above) sufficient to interfere in the replication and transmission of a wild-type coronavirus. A dose may be determined and/or adjusted within this range based on, e.g., age, physical condition, body weight, sex, diet, time of administration, and other clinical factors. A vaccine formulation may be systemically administered, e.g., by subcutaneous or intramuscular injection using a needle and syringe, or a needle-less injection device. Alternatively, a vaccine formulation may be administered intranasally, either by drops, large particle aerosol (greater than about 10 microns), or spray into the upper respiratory tract.

Method of Use

Another aspect of the present disclosure relates to a method of treating a subject infected with a coronaviridae virus. This method involves administering to said subject an effective amount of a pharmaceutical composition, as described above, comprising the DI coronaviridae virus-like particles as described herein in an amount effective to impair replication and spread of the coronaviridae virus in the subject.

As such, the present disclosure further provides a method of treating a coronavirus infection in an animal infected or suspected to be infected with a coronavirus infection, the method comprising administering at least one effective dose of a composition comprising DI coronaviridae virus-like particle as disclosed herein. The composition may be administered to a subject in any suitable manner, such as orally, intradermally, intramuscularly, intraperitoneally, intravenously, subcutaneously, or via pulmonary administration (e.g., through an inhaler).

Suitable subjects to be treated in accordance with the methods and compositions disclosed herein include, without limitation, mammalian subject. Exemplary mammalian subjects include, without limitation, humans, non-human primates, dogs, cats, rodents (e.g., mouse, rat, guinea pig), horses, cattle and cows, sheep, and pigs. In some embodiments, the subject is a human subject.

An effective amount of a composition comprising DI coronaviridae virus-like particles may be determined on a basis of number of virus-like particles or by weight of an expressed protein, such as one or more of the nsp proteins described above, ORF proteins, E protein, or N protein. For example, in any embodiment, a therapeutically effective amount may be about 0.04 μg, about 0.2 μg, about 0.4 μg, about 0.8 μg, about 0.5 μg, about 0.25 μg, about 1 μg, about 5 μg, about 10 μg, about 20 μg, about 25 μg, about 30 μg, about 50 μg, about 100 μg, about 125 μg, about 150 μg, or about 200 μg by weight of an expressed coronaviridae protein.

In any embodiment, the DI coronaviridae virus-like particles may be effective to treat a subject infected with a human coronavirus. In some embodiments, the subject is infected with human severe acute respiratory syndrome coronavirus 2. In some embodiments, the subject is a human subject.

Treatment of a subject infected with a coronavirus with the DI virus-like particles as described herein can result in a reduction or elimination of disease, symptom, virus concentration, or other undesired property in a subject relative to a control population (for example, same or similar viral infection but without treatment by the described methods and materials). The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, virus concentration, or other undesired property).

The disclosure further provides for a method of vaccinating a subject against a coronavirus infection, the method comprising administering to said subject a protection-inducing amount of a composition comprising DI coronaviridae virus-like particles. Again, the composition may be administered to a subject in any suitable manner, such as orally, intradermally, intramuscularly, intraperitoneally, intravenously, subcutaneously, or via pulmonary administration.

The following examples are provided to further describe some of the embodiments disclosed herein. The examples are intended to illustrate, not to limit, the disclosed embodiments. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Example 1: Construction of DI Coronaviridae Virus Recombinant Constructs

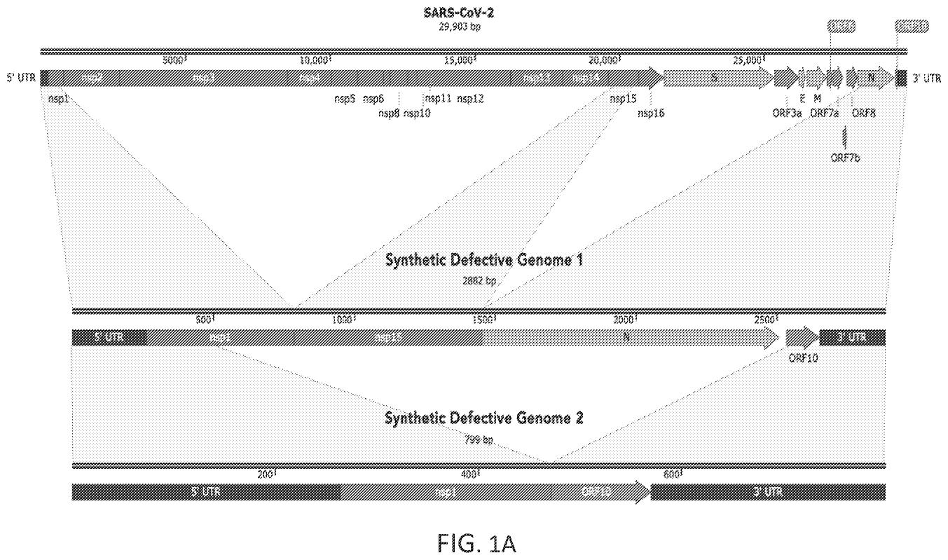

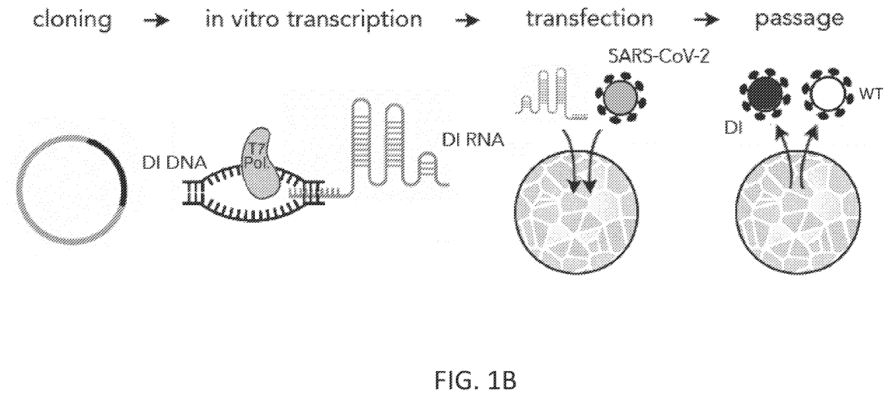

Examples of synthetic defective SARS-CoV-2 viruses constructed herein are shown schematically in FIG. 1a. The first version includes only the regions essential for replication (FIG. 1a, Synthetic Defective Genome 2). The second version includes, in addition to the replication signals, the packaging signals (FIG. 1a; Synthetic Defective Genome 1).

To demonstrate the ability of the synthetic defective viral RNA constructs to replicate and get packaged into viral capsids, a recombinant construct comprising the nucleotides sequence of SEQ ID NO: 31 as described supra was constructed and tested as described in Examples 2, 3, and 4 below.

### Example 2: Replication and Packaging of Synthetic Recombinant DI Coronaviridae RNAs

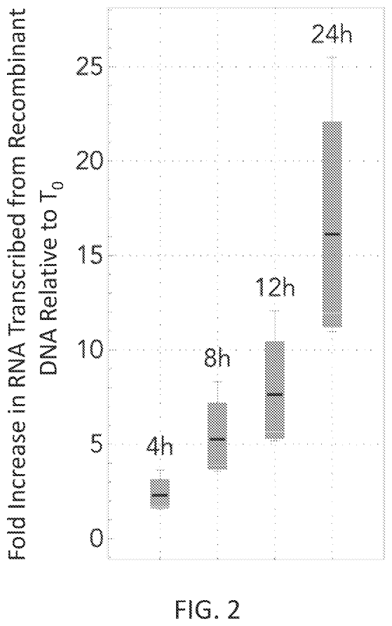

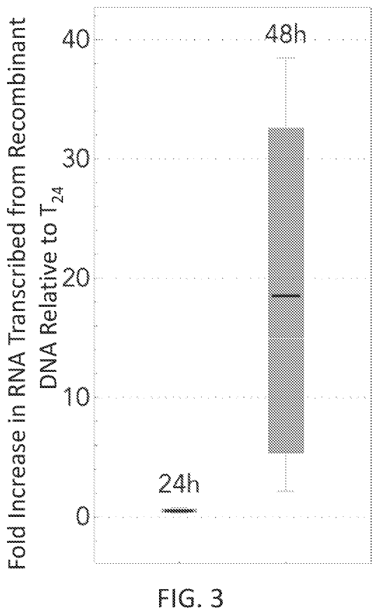

As noted above, a recombinant construct comprising the nucleotide sequence of SEQ ID NO: 31 was constructed for initial testing. In vitro transcription of this recombinant construct was carried out (using the mMESSAGE mMACHIN T7 Transcription Kit by Invitrogen, following the protocol provided by the manufacturer) to produce 2 μg of the corresponding synthetic RNA (i.e., SEQ ID NO: 32). The synthetic produced RNA was then transfected into 1 million Vero cells by electroporation (using a Lonza 4D nucleofector, following the protocol recommended by the manufacturer). The transfected Vero cells were then infected with SARS-CoV-2 (full length) at multiplicity of infection (MOI)=0.1 after 4 hours. FIG. 2 shows the increase (mean, 25% and 75% quartiles, upper and lower fences) of synthetic recombinant RNA as after 24 hours (T24) relative to the amount of RNA just after transfection (T₀), before infection with SARS-CoV-2, and at other time points (8, 12, 24 hours) after transfection. Fold increase of synthetic recombinant RNA was measured by qPCR and derived from the difference in replication cycles compared to a standard control gene (beta actin) as is standard procedure (Livak K J, Schmittgen T D, “Analysis of relative gene expression data using real-time quantitative PCR and the 2(-Delta Delta C(T)) Method,” Methods. 25(4):402-408 (2001), which is hereby incorporated by reference in its entirety). Note that by the time of infection (4 hours after transfection), the synthetic recombinant RNA increased by about 3 times compared to the amount of synthetic recombinant RNA at (T₀), indicating successful replication of the synthetic recombinant RNA. The increase in the amount of synthetic recombinant RNA is arguably much higher than 3-fold or even 4-fold, because cells were infected at an MOI of 0.1. Assuming a standard Poisson probability distribution, at an MOI of 0.1, less than 0.5% of the cells are co-infected by both SARS-CoV-2 virus and the synthetic recombinant RNAs at the 4-hour time point. The SARS-CoV-2 virus does not start to produce replication proteins until a few hours after infection. As more and more cells become infected with the full-length virus and are therefore co-infected with both full virus RNA and synthetic recombinant RNA, the RNAs degrade. At a higher MOI or if cells already infected by the full-length virus are transfected with synthetic recombinant RNA, the overall replication rate of the defective virus will be much higher, as shown by the increase of the RNA transcribed from the recombinant DNA constructs after 8, 12, and 24 hours relative to the amount of synthetic recombinant RNA at the same time point (24 hours after transfection) without SARS-CoV-2 (FIG. 2).

FIG. 3 shows the increase in the amount of synthetic recombinant RNA 24 hours after the supernatant is collected (24 hours after transfection of synthetic recombinant RNA) from cell cultures transfected with the RNA comprising SEQ ID NO: 32 and infected with the SARS-CoV-2 virus is used to infect new cells at an MOI of 0.1. The values are relative to the amount of synthetic recombinant RNA just after the new batch of cells is infected with the supernatant. Even at such a low MOI, the synthetic recombinant RNA continues to be detected 24 hours after the supernatant is passaged to new cells (that is, 48 hours after the initial transfection) (FIG. 3), suggesting that the synthetic recombinant RNA is being packaged and transmitted across cell batches in the presence of the full-length SARS-CoV-2 virus, and continues to replicate after spreading to new cells.

### Example 3: Synthetic Recombinant DI Coronavirida RNA Reduce the Amount of Viral RNA

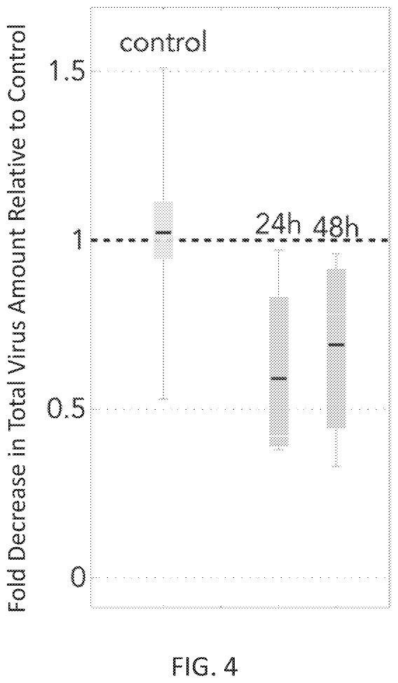

The amount of full-length wild type SARS-CoV-2 RNA in cells transfected with the synthetic recombinant RNA constructs as described in Example 2 was also measured by qPCR. FIG. 4 is a graph showing the fold decrease of SARS-CoV-2 RNA in the presence of the synthetic recombinant RNA constructs relative to the SARS-CoV-2 RNA at the same time point in control cell populations that were not transfected with synthetic recombinant RNA. This data suggests that the presence of the synthetic recombinant RNA leads to a significant reduction in the total amount of full-length SARS-CoV-2 virus, even when starting from, or passaging cells at, an MOI of 0.1 (as noted above, at this MOI, less than 0.5% of the cells are co-infected with SARS-CoV-2 virus and the synthetic recombinant RNA constructs). Given that the synthetic recombinant RNA is successfully and effectively packaged and transmitted across cell batches, this process will arguably continue and lead to a further decline in SARS-CoV-2 virus infection over time.

### Example 4: DI Coronaviridae Impairs Growth of Wild Type Virus

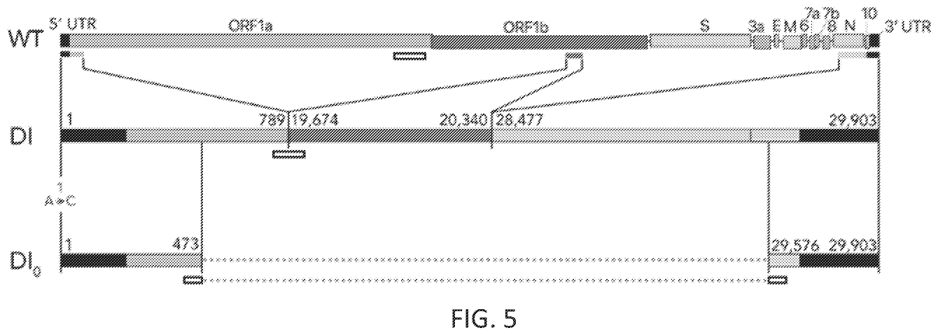

FIG. 5 depicts two recombinant nucleic acid constructs (DI, DI₀) that were constructed for this Example. Three segments of the wild type (WT) SARS-CoV-2 genome were used to create a synthetic DI genome and a shorter version of this DI genome (DI₀) comprising only parts of the two terminal segments. Numbers delimiting the segments refer to positions in the SARS-CoV-2 genome. The first position is mutated (A→C) in both DI and DI₀. Open rectangles show the position of the probes and primers used. The main synthetic construct (DI) is made from three segments: the 5′ UTR and the adjacent 5′ part of nsp1 in ORF1a; a part of nsp15 that includes the putative packaging signal; and the sequence spanning the 3′ part of the N sequence, ORF10 and the 3′UTR. The N fragment was chosen to include two of the most conserved regions of the virus genome. See, e.g., Rangan, et al., RNA. 2020; 26(8):937-959, which is incorporated herein by reference in its entirety. Because there is evidence that a long ORF enables DIs in certain coronaviruses (notably MHV (see, e.g., De Groot, et al., J Virol. 1992; 66(10):5898-5905, which is incorporated herein by reference in its entirety) which is closely related to SARS-CoV-2) to replicate more efficiently (even if coding for a chimeric non-functional protein (see, e.g., Van der Most, et al. Translation but not the encoded sequence is essential for the efficient propagation of the DI RNAs of the coronavirus mouse hepatitis virus. J Virol. 1995; 69(6):3744-3751, which is incorporated herein by reference in its entirety), the three fragments were assembled in frame, to retain a 2247nt ORF starting at the 5′ end of the sequence encoding nsp1 in FIG. 5; and because there is evidence that multiple transcriptional regulatory sequences (TRS) reduce replication efficiency (see, e.g., Joo & Makino, J Virol. 1995; 69(1):272-280; VanMarle, et al., J Virol. 1995; 69(12):7851-7856; Mendez, et al., Virology. 1996; 217(2):495-507, each of which is incorporated herein by reference in its entirety), the 3′ segment within the N sequence was chosen to start, to exclude its TRS. The synthetic sequence was analyzed to check for potential, aberrations in the RNA secondary structure.

Sequences and cloning. The DNA sequence of the DI genome (GenBank accession number: MW250351) was designed to correspond to the following three joint segments of the SARS-CoV-2 complete genome (the NCBI Reference Sequence for SARS-CoV-2; GenBank accession number: NC_045512.2), in the following order: 1 to 789; 19674 to 20340; and 28477 to 29903. The DI₀ genome (GenBank accession number: MW250350) was designed to correspond to the following two joint fragments of SARS-CoV-2 in the following order: 1 to 473; 29576 to 29903. In both cases, the first nucleotide of the first fragment was changed from A to C in order to improve in vitro transcription efficiency. The synthetic sequence was analyzed using the Vienna RNA package (Lorenz et al. ViennaRNA Package 2.0. Algorithms for Molecular Biology 2011; 6:1-26) to confirm the absence of potential aberrations in the RNA secondary structure. The DI and DI₀ genome DNA were assembled from synthetic oligonucleotides and inserted into a pMA-RQ plasmid by Invitrogen (Thermo Fisher Scientific). The T7 promoter (SEQ ID NO: 42) was synthetized immediately upstream of the 5′ end of the synthetic virus sequence. A short sequence (CCATGG) containing the NcoI restriction site was synthetized immediately upstream of the 5′ end of the T7 promoter, and a short sequence (CCGGT) containing the AgeI restriction site was synthetized immediately downstream of the 3′ end of the third fragment. The plasmid DNA was purified from transformed bacteria and the final construct was verified by sequencing.

In vitro transcription. The plasmid containing the synthetic DI or DI₀ genome DNA was linearized using NcoI and AgeI and re-suspended in H2O. 1 μg was then used as a template to produce capped RNA via T7 RNA polymerase, using a single reaction setup of the mMESSAGE mMACHINE® Kit (Applied Biosystems), which contains: 2 μL enzyme mix (buffered 50% glycerol containing RNA polymerase, RNase inhibitor, and other components); 2 μL reaction buffer (salts, buffer, dithiothreitol, and other ingredients); 10 μL of a neutralized buffered solution containing: 15 mM ATP, 15 mM CTP, 15 mM UTP, 3 mM GTP and 12 mM cap analog [m7G(5′)ppp(5′)G]; 4 μL nuclease-free H₂O; incubated for 2 hours at 37° C. RNA was isolated using TRIzol reagent (Invitrogen) extraction and isopropanol precipitation.

Cells and transfection. Vero-E6 cells (Chlorocebus sabaeus kidney epithelial cells cultured in DMEM medium (Hyclone, #SH30022.FS) supplemented with 10% fetal bovine serum (Corning, #35-011-CV), 100 units/mL penicillin and 100 μg/mL streptomycin (Gibco, #15140122) maintained at 37° C. and in a 5% CO₂ atmosphere were grown to 80% confluence. The cells were electroporated with the RNA produced by in vitro transcription (DI: 532ng; DI₀: 476 ng; per 200,000 cells; equivalent to 1.7×10⁶ and 5.6×10⁶ RNA molecules per cell, respectively), in 100 μl Nucleocuvette Vessels using the SF Cell solution and program DN-100 on a 4D Nucleofector X unit (Lonza). The efficiency of transfection was approximately 90%. Cells used for the control experiments were electroporated in the same way but without RNA.

Virus culture. SARS-CoV-2 isolate USA-WA1/2020 was obtained from BEI resources (#NR-52281) and propagated in Vero-E6 cells. Virus stocks were prepared and the titer as determined by plaque assays by serially diluting virus stock on Vero-E6 monolayers in the wells of a 24-well plate (Greiner bio-one, #662160). The plates were incubated at room temperature in a laminar flow hood with hand rocking every ten minutes. After one hour, an overlay medium containing 1×MEM, 1% Cellulose (Millipore Sigma, #435244), 2% FBS and 10 mM Hepes 7.5 was added and the plates were incubated for a further 48 hours at 37° C. The plaques were visualized by standard crystal violet staining. All work with the SARS-CoV-2 was conducted in Biosafety Level-3 conditions at the Eva J Pell Laboratory of Advanced Biological Research, The Pennsylvania State University, following the guidelines approved by the Institutional Biosafety Committee.

Coinfection and RNA extraction. 200,000 transfected cells were seeded in each well of a 24-well plate (each well in triplicate), and incubated for 1 hour before being inoculated with SARS-CoV-2 at an MOI of about 10. The medium containing the infectious SARS-CoV-2 viruses was removed after 1 hour and replaced with fresh medium. Cells were allowed to grow for 4, 8, 12 or 24 hours before RNA was extracted. The supernatant of cultures grown for 24 hours was used to infect new cells in 24-well plates for one hour, then media was replaced with fresh media and RNA was extracted from the cells after another 24 hours. This step was repeated four times to obtain RNA from four consecutive passages. RNA was extracted using Quick RNA miniprep kit (Zymo, #R1055) or TRIzol reagent (Invitrogen, #15596026) followed by isopropanol precipitation.

RNA analysis. Equal amounts of total RNA were reverse transcribed into first-strand cDNA using Revert Aid™ First Strand cDNA Synthesis Kit (Fermentas). 2 μL of diluted cDNA (3 pg-100 ng depending on the experiment) mixed with 2 μL of 5 μM primer mix (forward plus reverse, as shown in Table X below), 1 μL of 2 μM probe, and 5 μL master mix (2×) was used for qRT-PCR using TaqMan assay on a StepOnePlus instrument (Applied Biosystems) starting with polymerase activation at 95° C. for 3 minutes, followed by 40 cycles of denaturation (95° C., 15 seconds) and annealing/extension (60° C., 1 minute). The amount of WT and synthetic DI or DI₀ genome were quantified (using StepOnePlus Software 2.3) by the comparative CT method (Livak and Schmittgen, Methods. 2001; 25(4):402-408). All results were normalized with reference to the actin beta (ACTB) gene of Chlorocebus sabaeus. Each sample was repeated three times and the average value was used; all absolute values reported are 2^{-ΔCT} values. Primers and probes for the DI and DI₀ genomes were designed to amplify one of the junctions between segments of the WT genome. For the virus, a modified version of the CCDC primer-probe set on ORF1 was used. A BLAST search revealed no off-target sequences neither in the SARS-CoV-2 nor in the Chlorocebus sabaeus genome. Primers and probes for the DI and DI₀ genomes, for the SARS-CoV-2 genome, and for the ACTB gene of Vero-E6 cells, were labelled using the FAM dye, an IBFQ quencher and an additional internal (ZEN) quencher, and were synthetized by Integrated DNA Technologies. The sequences of these primers and probes are provided in Table 2 below.

TABLE 1

| | SEQ ID NO: | | |
|---|---|---|---|
| Construct | Forward | Reverse | Probe |
| DI | 43 | 44 | 45 |
| DI₀ | 46 | 47 | 48 |
| SARS-COV-2 | 49 | 50 | 51 |
| ACTB | 52 | 53 | 54 |

Mathematical model. The dynamics of intracellular competition between DI and WT were modeled using the following ordinary differential equation system:

$$\frac{{dx}_{WT}}{dt} = [ \frac{B}{- + {e}^{( - z ( t - {t}_{0} )}} ] [ \frac{1}{1 + {e}^{- s ( {x}_{WT} + 1 / n - h )}} - ( 1 - \frac{1}{R} ) ] {x}_{WT} - γ \cdot κ \cdot {x}_{-} \cdot {x}_{WT} - {δ}_{G} \cdot {x}_{WT} \qquad ( 1 )$$

$$\frac{{dx}_{DI}}{dt} = [ \frac{B}{- + {e}^{( - z ( t - {t}_{0} )}} ] [ \frac{1}{1 + {e}^{- s ( {x}_{WT} - h )}} ] {x}_{DI} - ω \cdot γ \cdot κ \cdot {x}_{-} \cdot {x}_{DI} - {δ}_{G} \cdot {x}_{DI} \qquad ( 2 )$$

$$\frac{{dx}_{C}}{dt} = [ \frac{B}{- + {e}^{( - z ( t - {t}_{0} )}} ] η \cdot {x}_{WT} - κ \cdot ( {x}_{WT} + ω \cdot {x}_{DI} ) \cdot {x}_{C} - {δ}_{C} \cdot {x}_{C} \qquad ( 3 )$$

The three equations (1-3) represent the change over time (t) of, respectively, the number of WT genomes (x_{WT}), of DI genomes (x_{DI}) and capsids (x_{C}). In each equation, the first term is the rate of increase due to replication; the second is the rate of loss due to encapsidation; the third is the rate of loss due to degradation. New WT and DI genomes are produced (first term of equations 1 and 2, respectively) at a rate given by a logistic function (with steepness s and inflection point h) of the number of WT genomes (as DI genomes do not produce any viral protein) multiplied by a factor corresponding to the amount of resource (B) within the cell, which is assumed to be time-dependent and changes at a rate given by a logistic function with negative steepness z and inflection point to (the time point when resources are depleted by half). WT genomes pay a cost equal to 1−1/R, where R is the ratio between the rate of replication of DI and WT (R>1 given that the DI genome is shorter and can replicate faster) but have a slight advantage due to the additional viral genome (itself) producing replication proteins among the n neighbouring genomes. n is assumed to be large enough to ignore the variance in the number of WT genomes around n (which, in a large population, would be binomially distributed). WT and DI genomes decrease as a function of their decay rate δ_{G} and the rate of encapsidation (κ, the rate of encapsidation for the WT genome; ω, the ratio between the rate of encapsidation of DI and WT; and γ, number of genomes per capsids). The number of capsids (equation 3) increase as a linear function (controlled by the capsid/genome ratio η) of the number of WT genomes and decreases as a function of the number of encapsidated genomes; and as a function of the decay rate δ_{C}.

Results: The length of the synthetic DNA construct DI is 2882 nt, which is 9.6% of the full-length genome (29903 nt). A shorter (800 nt) defective genome (DI₀) was synthesized without the second segment (the putative packaging signal) and with shorter terminal segments, as shown in FIG. 5. The DI and DI₀ genomes, synthetized as DNA and inserted into plasmids, were transcribed in vitro to yield genomic RNAs, which were then electroporated in Vero-E6 cells that were infected with SARS-CoV-2.

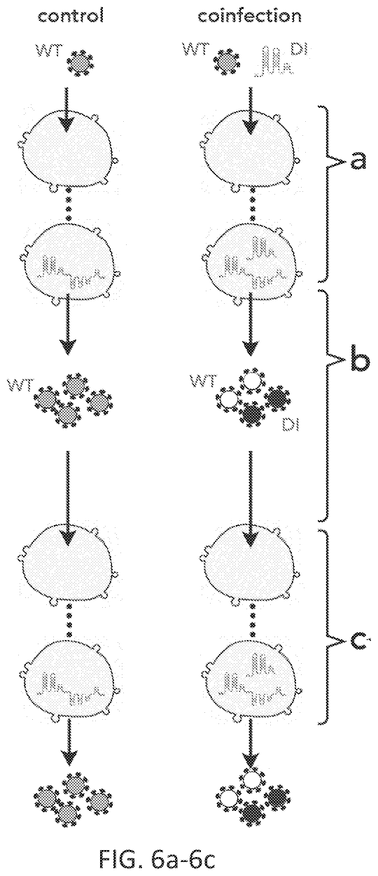

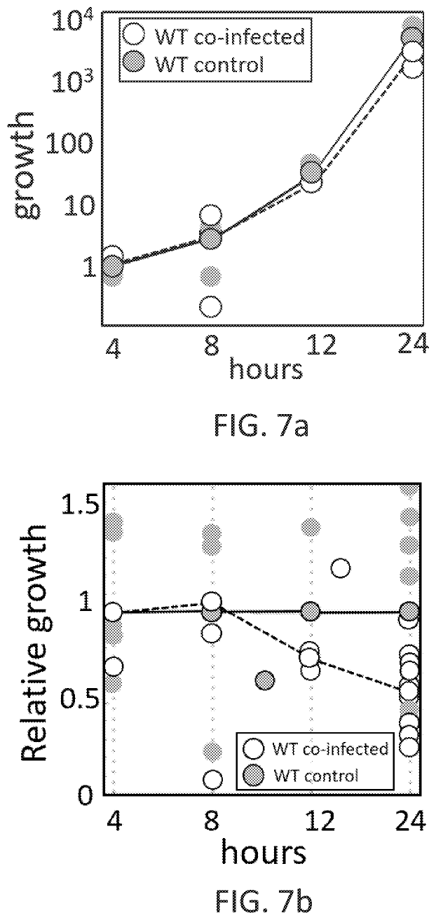

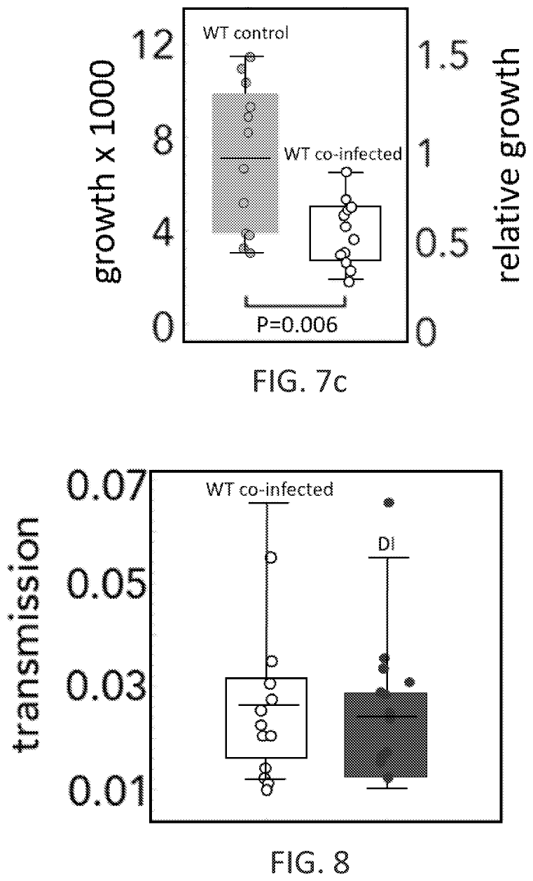

A schematic of a timeline of replication of the WT SARS-CoV-2 virus alone (control) or when coinfected with DI construct in Vero-E6 cells (cotransfection) is shown in FIG. 6. Because of the large amount of synthetic RNA transfected, the fast degradation of the synthetic RNA inside cells (in the absence of the virus, 1% to 4% of the synthetic RNA can be detected by qRT-PCR 4 hours post transfection) and the lag between infection and the start of replication, it is not possible to quantify the replication rate of the DI and DI₀ genomes, or even prove their replication, immediately after RNA transfection, as most of the RNA cannot replicate and will simply be degraded. It is possible, however, to quantify its effect on virus replication: the DI genome reduced the amount of SARS-CoV-2 by approximately half (compared to the amount of virus in control experiments) within 24 hours of transfection, shown in FIGS. 7a, 7b, and 7c, which corresponds to stage a in FIG. 6; the DI₀ genome had no significant effect.

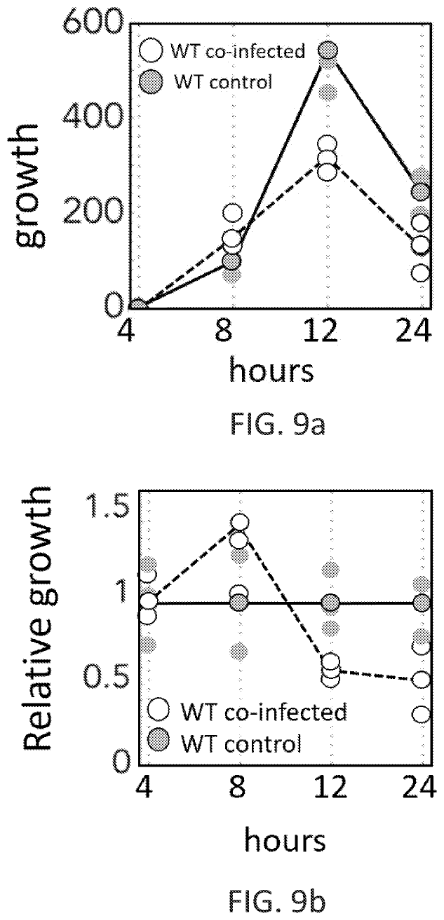

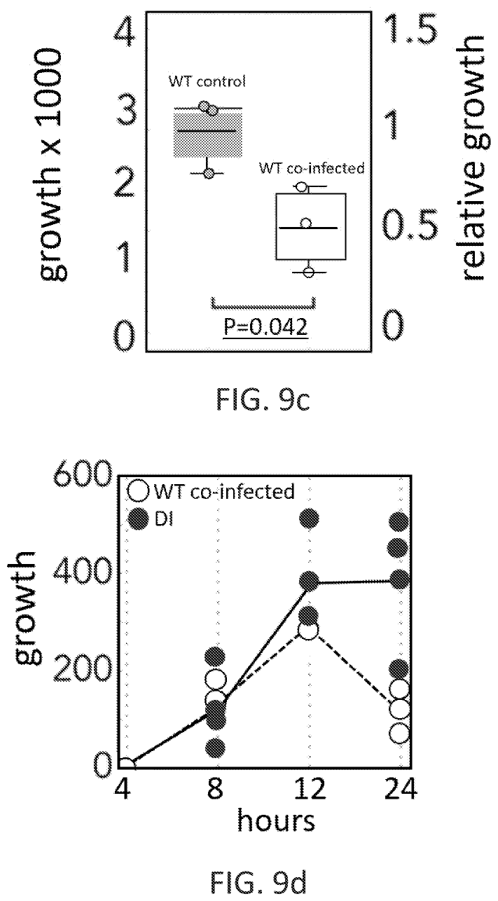

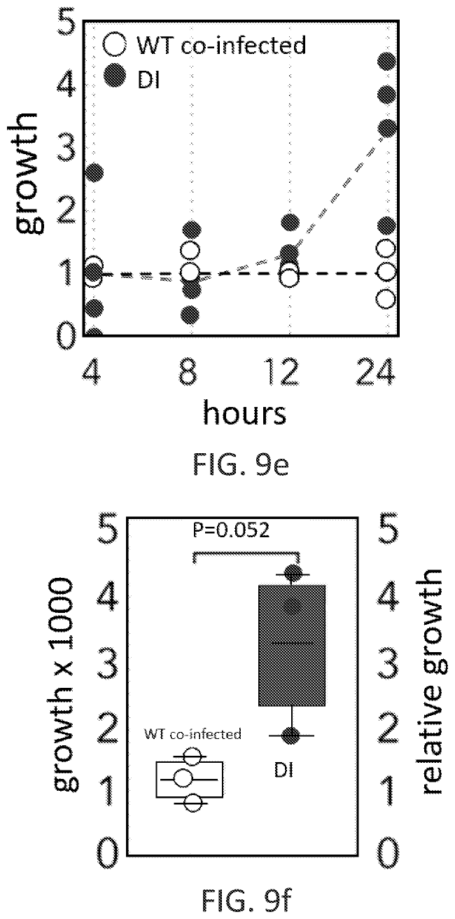

24 hours post transfection the supernatants were collected and used to infect new cell monolayers (stage b of FIG. 6). In these cells, the DI and WT genomes were detected (by qRT-PCR) 4 to 24 hours after the transfer. The DI₀ genome was not detected, suggesting that the middle fragment of the DI synthetic sequence does have a positive effect on packaging. The transmission rate of the DI virus does not differ from that of the WT virus, as shown in FIG. 8 (corresponding to stage b in FIG. 6), suggesting that the synthetic genome gets packaged into virus-like particles with essentially the same efficiency as the full-length virus, and that these virus-like particles are as infectious. In the cells co-infected by DI and SARS-CoV-2, the WT genome again declines by approximately half in 24 hours as shown in FIGS. 9a-9c, which corresponds to stage c in FIG. 6. The replication rate of the DI genome can now be quantified, revealing that it increases 3.3 times as fast as the WT virus (since the supernatant from the previous passage was removed 1 hour after infection, the increase observed must be due to replication) (see FIGS. 9d-9f).

The supernatant was transferred to new cells that had been co-infected with the WT virus, after 24 hours, repeating the transfer to new cells four times. The DI genome was detected across all four passages and the DI/WT ratio increased approximately 3 times every 24 hours, consistent with the relative replication advantage and equal transmission efficiency measured. The absolute WT/DI ratio was unable to be measured because the amount of DI was below the level detectable by digital PCR. Results, therefore, suggest that the interference of even a small amount of DI can have a strong impact on the replication of the WT virus and its burden for the cell.

To study the dynamics of the system, a mathematical model of intra-cell competition between WT and DI genomes was used. The model is inspired by a similar model by Shirogane et al., bioRxiv 2019; 519751 (which is incorporated herein by reference in its entirety), and is used to describe replication-independent poliovirus DIs. In the instant model, however, replication of the DI is entirely dependent on the WT. Here the same approach is used but a further step is taken in assuming that the replication of the DIs depends on the amount of proteins produced by multiple genomes and that these have a nonlinear effect on replication.

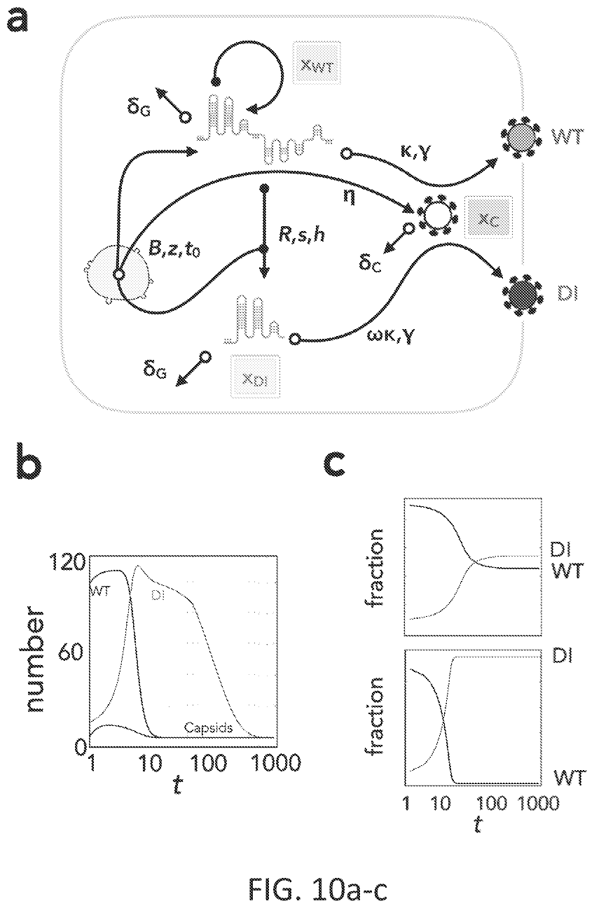

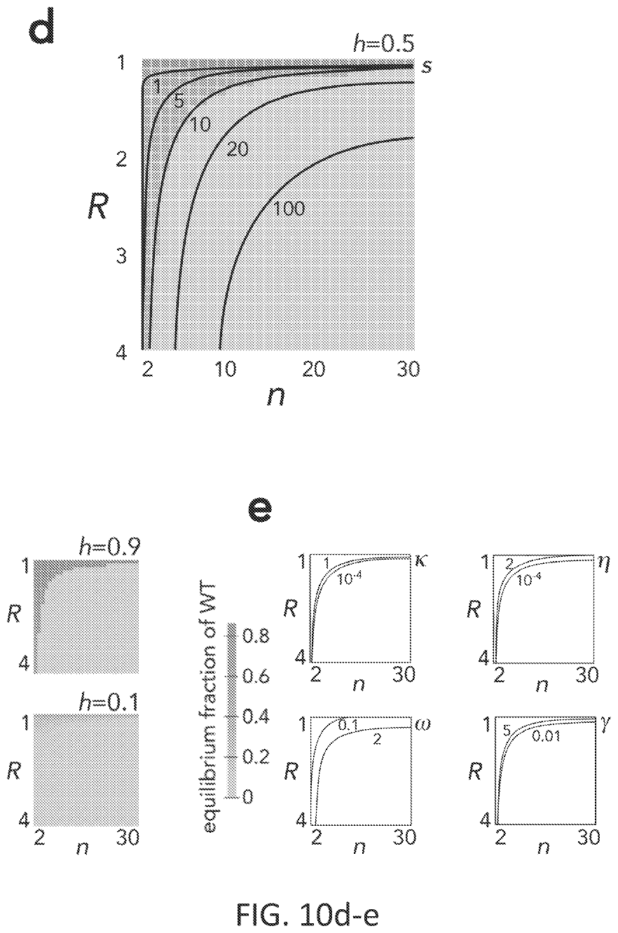

As predicted by early models of DI-WT dynamics (such as those by Szathmiry, J Theor Biol. 1994; 157(3):383-406, which is incorporated herein by reference in its entirety) and by the general theory of non-liner public goods (such as that by Archetti, Games 2018; 9:17, which is incorporated herein by reference in its entirety), a polymorphic equilibrium exists, in which DI and WT coexist, if the replication advantage (R) of the DI genome is below a critical threshold, whereas above that threshold DI drives WT to extinction. The critical threshold depends mainly on the number of genomes within the range of the viral protein produced by the WT genome (n). Other parameters, including the ones that control resource depletion, encapsidation and spontaneous decay, are less important for the dynamics (at the value of R measured, for realistic values of n, they are almost irrelevant). While most of these parameters remain to be measured, therefore, the model suggests that the DI genome will, given the high replication advantage measured, increase in frequency over time, eventually leading to the extinction of the WT genome. As coinfections were monitored for only 5 passages, however, whether the slight increase in DI/WT ratio that observed in coinfections is indeed the early stage of a reduction that would lead to the extinction of the WT virus was unable to be verified. Simulation of the dynamics of DI-WT competition is shown in FIGS. 10a-10e as follows: FIG. 10a: Flow diagram of the model. The number of WT genomes (x_{WT}), DI genomes (x_{DI}) and capsids (x_{C}) increase due production and decline due to degradation and encapsidation. Production is proportional to the amount of resources of the cell (B), which decreases as a logistic function (with steepness z and inflection t₀) of time (t); and increases as a linear function (for capsids) or as a logistic function (with steepness s and inflection h) of the number of WT genomes (for WT and DI genomes). DI genomes replicates at a rate R relative to WT genomes. Genomes decay at a rate δ_{G}; capsids decay at a rate δ_{C}. The rates of encapsidation are κ for WT genomes and ωκ for DI genomes; γ is the number of genomes per capsids; η is the capsid/Genome ratio. FIG. 10b: Example of the results: number of WT and DI genomes and capsids over time. The change in the amount of DI, WT and capsids over time for R=3.3 (the value measures empirically); other parameters: n=10; s=10; h=0.5; δ_{G}=0.01; δ_{C}=0.001; γ=1; η=0.3; κ=0.03, ω=0.9; B=2, z=−0.03; t₀=10; starting from 100 WT and 10 DI genomes. FIG. 10c: Examples of the results: fraction of WT and DI genomes over time. The change in frequency of DI and WT over time for two values of R: R=3.3 (left; the value measured empirically) or R=1.3 (right) assuming no depletion of resources (B=2, z=0); other parameters: n=10; s=10; h=0.5; δ_{G}=0.01; δ_{C}=0.001; γ=1; η=0.3; κ=0.03, ω=0.9. The initial fraction of DI in this example is 0.1, but the results are independent from the initial fraction. FIG. 10d: Summary of the effect of R and n on the results. The color of each cell shows the stable fraction of DI as a function of the Replicat® advantage (R) of the DI genome and the number of genomes within the range of the viral protein produced by the WT genome (n), for different values of h; assuming no resource depletion (B=2, z=0); other parameters: s=10; δ_{G}=0.01; δ_{C}=0.001; γ=1; η=0.3; κ=0.03, ω=0.9. For h=0.5 the curves show, for different values of s from 1 to 100, the separation between the combination of parameters for which WT goes extinct (below the curve) or remains at a stable polymorphic equilibrium (above). FIG. 10e: Summary of the effect of other parameters on the results. Combinations of R and n below the curves lead to the extinction of WT. The curves are drawn for different values of γ, η, κ and ω; other parameters now shown because differences are undetectable. In all cases, h=0.5, s=10.

DI particles have long been known to virologists and their use in unravelling the location of functional elements of a genome is well known. Here a synthetic DIs was used to show that a disputed putative packaging sequence of SARS-CoV-2 does indeed enable packaging of the synthetic genome—and therefore presumably acts as a packaging signal for the WT genome as well. However, because the difference between the DI and DI₀ synthetic constructs is not limited to the segment with the putative packaging signal, it cannot be ruled out that the packaging signals resides in the other parts of DI that DI₀ lacks, most notably a conserved region (28554 . . . 28569) with a SL5 motifs in the N partial sequence included in the DI genome but not in the DI₀ genome.

The interference with the WT virus is, however, the most remarkable effect of the DI construct disclosed herein. DI particles are often described as by-products of inefficient replication or as having a regulatory function. Seen, instead, as defectors in the sense of evolutionary game theory, they need not serve any purpose for the WT virus: they exist as ultra-selfish replicators, able to free-ride as parasites of the full-length genome. As such, DI particles have a potential as antivirals: by virtue of their faster replication in cells coinfected with the virus, DI genomes can replicate faster and, in the process, interfere with the WT virus. As the DI genomes increase in frequency among the virus particles pool, the process becomes more and more effective, until the reduction in the amount of WT virus leads to the demise of both virus and DI. By enabling the replication and spread of the DI genome, the virus effectively promotes its own demise. The potential of DIs as antivirals has been suggested, but not yet exploited, for other viruses (Marriott & Dimmock, Rev Med Virol. 2010; 20:51-62; Dimmock & Easton J Virol. 2014; 88(10):5217-5227; Vignuzzi & López Nat Microbiol. 2019; 4(7):1075-1087, which are hereby incorporated by reference in their entirety), and a similar therapeutic approach has been proposed for bacteria (Brown et al. Phil. Trans. R. Soc. B 2009; 364:3157-3168, which is hereby incorporated by reference in its entirety) and cancer (Archetti, Evolution, Medicine and Public Health 2013; 1:161-172; Archetti et al. PNAS 2015; 112:1833-1838, which are hereby incorporated by reference in their entirety). While viruses such as HIV and influenza where DI therapy has been attempted (Marriott & Dimmock, Rev Med Virol. 2010; 20:51-62; Dimmock & Easton, J Virol. 2014; 88(10):5217-5227; Vignuzzi & López, Nat Microbiol. 2019; 4(7):1075-1087, which are hereby incorporated by reference in their entirety) are not ideal for this approach, because of the short genome, multiple genomic fragments and complex life cycles, coronaviruses are ideal candidates because of their long single strand RNA genome and relatively simple replication process. A version of our synthetic DI could be used as an antiviral that would be self-sustaining and evolution-proof, or as a self-disseminating approach to suppress zoonoses (Nuismer & Bull, Nat Ecol Evol 2020; 4, 1168-1173, which is hereby incorporated by reference in its entirety) that could spill over to humans in the future.

### Example 5: Interference of WT SARS-CoV-2 with Synthetic DI RNA Construct Delivered Via Nanoparticles or Lipofection

In Examples 1-4 above, the synthetic recombinant RNA constructs were delivered by nucleofection, which is not an ideal method for therapeutic delivery. Accordingly, to test efficacy of synthetic recombinant RNA constructs delivered via a method suitable for therapeutic use in clinical environments, recombinant RNA constructs were delivered by polymer nanoparticles according to the methods disclosed in U.S. Patent Application Publication No. US2019/0125874A1, which is incorporated herein by reference in its entirety or lipofection. All experiments were carried out with Vero-E6 cells, infected at an MOI of 10; DI RNA, when used, was used at 60 ng per well (200,000 cells).

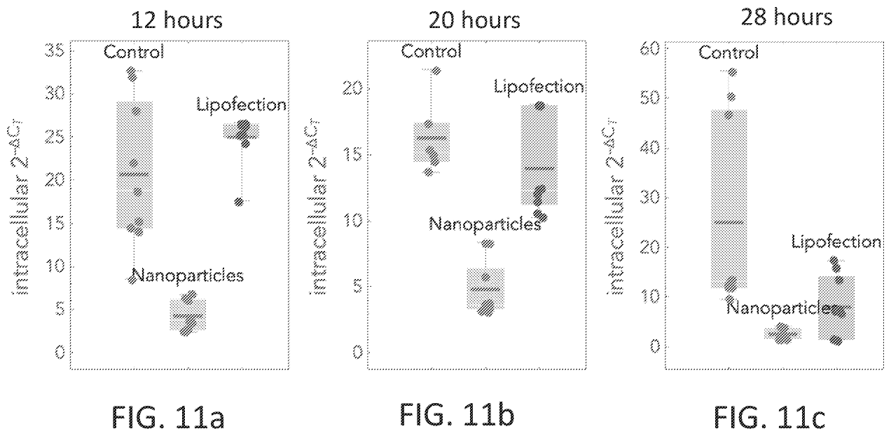

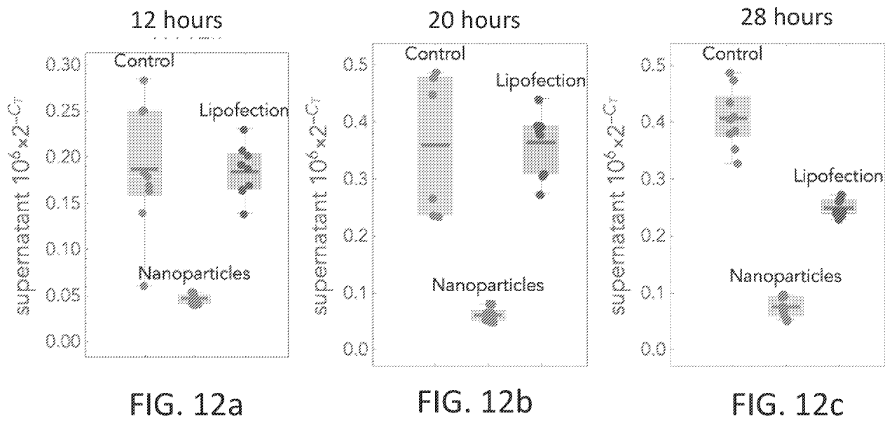

Three treatment groups were studied: 1) control: which consisted of SARS-CoV-2 infected cells only; 2) nanoparticles: SARS-CoV-2 in the presence of synthetic recombinant RNA encoding DI virus delivered via polymer nanoparticles; 3) lipofection: SARS-CoV-2 in the presence of synthetic recombinant RNA encoding DI virus delivered via lipofection (Lipofectamine™ 2000, Invitrogen). Levels of intracellular WT SARS-CoV-2 (FIGS. 11a-11c) and WT SARS-CoV-2 in the supernatants (FIGS. 12a-12c) was measured at 12, 20, and 28 hours after delivery. Results, as shown in FIGS. 11 and 12 indicate that the synthetic recombinant RNA encoding DI virus delivered via polymer nanoparticles effectively reduces the amount of intracellular SARS-CoV-2 WT virus to 18% (compared to infections with SARS-CoV-2 WT virus controls without DI) after 12 hours (FIG. 11a), and reduced further to 10% of at 28 hours (FIG. 11c).

Results are similar in the supernatant, as can be seen in FIGS. 12a-12c. Synthetic recombinant RNA encoding DI corona virus effectively appears to reduce the amount of WT SARS-CoV-2 (faster when delivered via polymer nanoparticles than via lipofection). Note that, while the level of WT SARS-CoV-2 doubles in the control treatment group over the 28 hours, the level of WT SARS-CoV-2 remains the same in the presence of DI, which indicates interference.

In summary, delivery by nanoparticles improves the efficacy of the synthetic recombinant nucleic acid sequences encoding DI virus-like particles, as described herein, to interfere in a coronavirus infection, specifically, an infection of SARS-CoV-2 and therefore may be used as a self-disseminating against a coronavirus infection. The DI virus replicate three times faster than SARS-CoV-2 in co-infected cells, is transmitted with the same efficiency as SARS-CoV-2, and reduces viral load of SARS-CoV-2 within cells by half within 24 hours. Moreover, this makes the synthetic DI RNA suitable for delivery in vivo to patients, as these nanoparticles conjugated with RNA have been shown to be able to reach the epithelial cells of the lungs after nebulization. See, e.g., Patel, A K, et al., Adv Mater. 2019 February; 31(8), which is incorporated herein by reference in its entirety.

TABLE 2
Sequences of SARS-COV-2 Proteins and Defective Interfering
Viruses as Disclosed Herein

| SEQ | | |
|---|---|---|
| ID NO: | Description | Sequence |
| 1 | 5′UTR of | attaaaggtt tataccttcc caggtaacaa accaaccaac tttcgatctc ttgtagatct |
| | SARS-CoV-2 | gttctctaaa cgaactttaa aatctgtgtg gctgtcactc ggctgcatgc ttagtgcact |
| | | cacgcagtat aattaataac taattactgt cgttgacagg acacgagtaa ctcgtctatc |
| | | ttctgcaggc tgcttacggt ttcgtccgtg ttgcagccga tcatcagcac atctaggttt |
| | | cgtccgggtg tgaccgaaag gtaag |
| 2 | 5′UTR of | auuaaagguu uauaccuucc cagguaacaa accaaccaac uuucgaucuc uuguagaucu |
| | SARS-CoV-2 | guucucuaaa cgaacuuuaa aaucugugug gcugucacuc ggcugcaugc uuagugcacu |
| | | cacgcaguau aauuaauaac uaauuacugu cguugacagg acacgaguaa cucgucuauc |
| | | uucugcaggc ugcuuacggu uucguccgug uugcagccga ucaucagcac aucuagguuu |
| | | cguccgggug ugaccgaaag guaag |
| 3 | 3′UTR of | caatct |
| | SARS-CoV-2 | ttaatcagtg tgtaacatta gggaggactt gaaagagcca ccacattttc accgaggcca |
| | | cgcggagtac gatcgagtgt acagtgaaca atgctaggga gagctgccta tatggaagag |
| | | ccctaatgtg taaaattaat tttagtagtg ctatccccat gtgattttaa tagcttctta |
| | | ggagaatgac aaaaaaaaaa aaaaaaaaaa aaaaaaaaaa aaa |
| 4 | 3′UTR of | caaucuuuaa ucagugugua acauuaggga ggacuugaaa gagccaccac auuuucaccg |
| | SARS-CoV-2 | aggccacgcg gaguacgauc gaguguacag ugaacaaugc uagggagagc ugccuauaug |
| | | gaagagcccu aauguguaaa auuaauuuua guagugcuau ccccauguga uuuuaauagc |
| | | uucuuaggag aaugacaaaa aaaaaaaaaa aaaaaaaaaa aaaaaaaaa |
| 5 | nsp1 from | atgga |
| | SARS-CoV-2 | gagccttgtc cctggtttca acgagaaaac acacgtccaa ctcagtttgc ctgttttaca |
| | | ggttcgcgac gtgctcgtac gtggctttgg agactccgtg gaggaggtct tatcagaggc |
| | | acgtcaacat cttaaagatg gcacttgtgg cttagtagaa gttgaaaaag gcgttttgcc |
| | | tcaacttgaa cagccctatg tgttcatcaa acgttcggat gctcgaactg cacctcatgg |
| | | tcatgttatg gttgagctgg tagcagaact cgaaggcatt cagtacggtc gtagtggtga |
| | | gacacttggt gtccttgtcc ctcatgtggg cgaaatacca gtggcttacc gcaaggttct |
| | | tcttcgtaag aacggtaata aaggagctgg tggccatagt tacggcgccg atctaaagtc |
| | | atttgactta ggcgacgagc ttggcactga tccttatgaa gattttcaag aaaactggaa |
| | | cactaaacat agcagtggtg ttacccgtga actcatgcgt gagcttaacg gaggg |
| 6 | nsp1 from | auggagagcc uugucccugg uuucaacgag aaaacacacg uccaacucag uuugccuguu |
| | SARS-CoV-2 | uuacagguuc gcgacgugcu cguacguggc uuuggagacu ccguggagga ggucuuauca |
| | | gaggcacguc aacaucuuaa agauggcacu uguggcuuag uagaaguuga aaaaggcguu |
| | | uugccucaac uugaacagcc cuauguguuc aucaaacguu cggaugcucg aacugcaccu |
| | | cauggucaug uuaugguuga gcugguagca gaacucgaag gcauucagua cggucguagu |
| | | ggugagacac uugguguccu ugucccucau gugggcgaaa uaccaguggc uuaccgcaag |
| | | guucuucuuc guaagaacgg uaauaaagga gcugguggcc auaguuacgg cgccgaucua |
| | | aagucauuug acuuaggcga cgagcuuggc acugauccuu augaagauuu ucaagaaaac |
| | | uggaacacua aacauagcag ugguguuacc cgugaacuca ugcgugagcu uaacggaggg |
| 7 | Nsp1 | MESLVPGFNE KTHVQLSLPV LQVRDVLVRG FGDSVEEVLS EARQHLKDGT CGLVEVEKGV |
| | | LPQLEQPYVF IKRSDARTAP HGHVMVELVA ELEGIQYGRS GETLGVLVPH VGEIPVAYRK |
| | | VLLRKNGNKG AGGHSYGADL KSFDLGDELG TDPYEDFQEN WNTKHSSGVT RELMRELNGG |
| 8 | ORF10 from | atg |
| | SARS-CoV-2 | ggctatataa acgttttcgc ttttccgttt acgatatata gtctactctt gtgcagaatg |
| | | aattctcgta actacatagc acaagtagat gtagttaact ttaatctcac atag |
| 9 | ORF10 from | augggcuaua uaaacguuuu cgcuuuuccg uuuacgauau auagucuacu cuugugcaga |
| | SARS-CoV-2 | augaauucuc guaacuacau agcacaagua gauguaguua acuuuaaucu cacauag |
| 10 | ORF10 | MGYINVFAFP FTIYSLLLCR MNSRNYIAQV DVVNFNLT |
| 11 | nsp15 from | agtttagaaa atgtggcttt taatgttgta aataagggac actttgatgg acaacagggt |
| | SARS-CoV-2 | gaagtaccag tttctatcat taataacact gtttacacaa aagttgatgg tgttgatgta |
| | | gaattgtttg aaaataaaac aacattacct gttaatgtag catttgagct ttgggctaag |
| | | cgcaacatta aaccagtacc agaggtgaaa atactcaata atttgggtgt ggacattgct |
| | | gctaatactg tgatctggga ctacaaaaga gatgctccag cacatatatc tactattggt |
| | | gtttgttcta tgactgacat agccaagaaa ccaactgaaa cgatttgtgc accactcact |
| | | gtcttttttg atggtagagt tgatggtcaa gtagacttat ttagaaatgc ccgtaatggt |
| | | gttcttatta cagaaggtag tgttaaaggt ttacaaccat ctgtaggtcc caaacaagct |
| | | agtcttaatg gagtcacatt aattggagaa gccgtaaaaa cacagttcaa ttattataag |
| | | aaagttgatg gtgttgtcca acaattacct gaaacttact ttactcagag tagaaattta |
| | | caagaattta aacccaggag tcaaatggaa attgatttct tagaattagc tatggatgaa |
| | | ttcattgaac ggtataaatt agaaggctat gccttcgaac atatcgttta tggagatttt |
| | | agtcatagtc cgttaggtgg tttacatcta ctgattggac tagctaaacg ttttaaggaa |
| | | tcaccttttg aattagaaga ttttattcct atggacagta cagttaaaaa ctatttcata |
| | | acagatgcgc aaacaggttc atctaagtgt gtgtgttctg ttattgattt attacttgat |
| | | gattttgttg aaataataaa atcccaagat ttatctgtag tttctaaggt tgtcaaagtg |
| | | actattgact atacagaaat ttcatttatg ctttggtgta aagatggcca tgtagaaaca |
| | | ttttacccaa aattacaa |
| 12 | nsp15 from | aguuuagaaa auguggcuuu uaauguugua aauaagggac acuuugaugg acaacagggu |
| | SARS-CoV-2 | gaaguaccag uuucuaucau uaauaacacu guuuacacaa aaguugaugg uguugaugua |
| | | gaauuguuug aaaauaaaac aacauuaccu guuaauguag cauuugagcu uugggcuaag |
| | | cgcaacauua aaccaguacc agaggugaaa auacucaaua auuugggugu ggacauugcu |
| | | gcuaauacug ugaucuggga cuacaaaaga gaugcuccag cacauauauc uacuauuggu |
| | | guuuguucua ugacugacau agccaagaaa ccaacugaaa cgauuugugc accacucacu |
| | | gucuuuuuug augguagagu ugauggucaa guagacuuau uuagaaaugc ccguaauggu |
| | | guucuuauua cagaagguag uguuaaaggu uuacaaccau cuguaggucc caaacaagcu |
| | | agucuuaaug gagucacauu aauuggagaa gccguaaaaa cacaguucaa uuauuauaag |
| | | aaaguugaug guguugucca acaauuaccu gaaacuuacu uuacucagag uagaaauuua |
| | | caagaauuua aacccaggag ucaaauggaa auugauuucu uagaauuagc uauggaugaa |
| | | uucauugaac gguauaaauu agaaggcuau gccuucgaac auaucguuua uggagauuuu |
| | | agucauaguc aguuaggugg uuuacaucua cugauuggac uagcuaaacg uuuuaaggaa |
| | | ucaccuuuug aauuagaaga uuuuauuccu auggacagua caguuaaaaa cuauuucaua |
| | | acagaugcgc aaacagguuc aucuaagugu guguguucug uuauugauuu auuacuugau |
| | | gauuuuguug aaauaauaaa aucccaagau uuaucuguag uuucuaaggu ugucaaagug |
| | | acuauugacu auacagaaau uucauuuaug cuuuggugua aagauggcca uguagaaaca |
| | | uuuuacccaa aauuacaa |
| 13 | nsp15 | SLENVAFNVV NKGHFDGQQG EVPVSIINNT VYTKVDGVDV ELFENKTTLP VNVAFELWAK |
| | | RNIKPVPEVK ILNNLGVDIA ANTVIWDYKR DAPAHISTIG VCSMTDIAKK PTETICAPLT |
| | | VFFDGRVDGQ VDLFRNARNG VLITEGSVKG LQPSVGPKQA SLNGVTLIGE AVKTQFNYYK |
| | | KVDGVVQQLP ETYFTQSRNL QEFKPRSQME IDFLELAMDE FIERYKLEGY AFEHIVYGDF |
| | | SHSQLGGLHL LIGLAKRFKE SPFELEDFIP MDSTVKNYFI TDAQTGSSKC VCSVIDLLLD |
| | | DFVEIIKSQD LSVVSKVVKV TIDYTEISFM LWCKDGHVET FYPKLQ |
| 14 | N protein from | atgtctg |
| | SARS-CoV-2 | ataatggacc ccaaaatcag cgaaatgcac cccgcattac gtttggtgga ccctcagatt |
| | | caactggcag taaccagaat ggagaacgca gtggggcgcg atcaaaacaa cgtcggcccc |
| | | aaggtttacc caataatact gcgtcttggt tcaccgctct cactcaacat ggcaaggaag |
| | | accttaaatt ccctcgagga caaggcgttc caattaacac caatagcagt ccagatgacc |
| | | aaattggcta ctaccgaaga gctaccagac gaattcgtgg tggtgacggt aaaatgaaag |
| | | atctcagtcc aagatggtat ttctactacc taggaactgg gccagaagct ggacttccct |
| | | atggtgctaa caaagacggc atcatatggg ttgcaactga gggagccttg aatacaccaa |
| | | aagatcacat tggcacccgc aatcctgcta acaatgctgc aatcgtgcta caacttcctc |
| | | aaggaacaac attgccaaaa ggcttctacg cagaagggag cagaggcggc agtcaagcct |
| | | cttctcgttc ctcatcacgt agtcgcaaca gttcaagaaa ttcaactcca ggcagcagta |
| | | ggggaacttc tcctgctaga atggctggca atggcggtga tgctgctctt gctttgctgc |
| | | tgcttgacag attgaaccag cttgagagca aaatgtctgg taaaggccaa caacaacaag |
| | | gccaaactgt cactaagaaa tctgctgctg aggcttctaa gaagcctcgg caaaaacgta |
| | | ctgccactaa agcatacaat gtaacacaag ctttcggcag acgtggtcca gaacaaaccc |
| | | aaggaaattt tggggaccag gaactaatca gacaaggaac tgattacaaa cattggccgc |
| | | aaattgcaca atttgccccc agcgcttcag cgttcttcgg aatgtcgcgc attggcatgg |
| | | aagtcacacc ttcgggaacg tggttgacct acacaggtgc catcaaattg gatgacaaag |
| | | atccaaattt caaagatcaa gtcattttgc tgaataggca tattgacgca tacaaaacat |
| | | tcccaccaac agagcctaaa aaggacaaaa agaagaaggc tgatgaaact caagccttac |
| | | cgcagagaca gaagaaacag caaactgtga ctcttcttcc tgctgcagat ttggatgatt |
| | | tctccaaaca attgcaacaa tccatgagca gtgctgactc aactcaggcc taa |
| 15 | N protein from | augucugaua auggacccca aaaucagcga aaugcacccc gcauuacguu ugguggaccc |
| | SARS-CoV-2 | ucagauucaa cuggcaguaa ccagaaugga gaacgcagug gggcgcgauc aaaacaacgu |
| | | cggccccaag guuuacccaa uaauacugcg ucuugguuca ccgcucucac ucaacauggc |
| | | aaggaagacc uuaaauuccc ucgaggacaa ggcguuccaa uuaacaccaa uagcagucca |
| | | gaugaccaaa uuggcuacua ccgaagagcu accagacgaa uucguggugg ugacgguaaa |
| | | augaaagauc ucaguccaag augguauuuc uacuaccuag gaacugggcc agaagcugga |
| | | cuucccuaug gugcuaacaa agacggcauc auauggguug caacugaggg agccuugaau |
| | | acaccaaaag aucacauugg cacccgcaau ccugcuaaca augcugcaau cgugcuacaa |
| | | cuuccucaag gaacaacauu gccaaaaggc uucuacgcag aagggagcag aggcggcagu |
| | | caagccucuu cucguuccuc aucacguagu cgcaacaguu caagaaauuc aacuccaggc |
| | | agcaguaggg gaacuucucc ugcuagaaug gcuggcaaug gcggugaugc ugcucuugcu |
| | | uugcugcugc uugacagauu gaaccagcuu gagagcaaaa ugucugguaa aggccaacaa |
| | | caacaaggcc aaacugucac uaagaaaucu gcugcugagg cuucuaagaa gccucggcaa |
| | | aaacguacug ccacuaaagc auacaauguu acacaagcuu ucggcagacg ugguccagaa |
| | | caaacccaag gaaauuuugg ggaccaggaa cuaaucagac aaggaacuga uuacaaacau |
| | | uggccgcaaa uugcacaauu ugcccccagc gcuucagcgu ucuucggaau gucgcgcauu |
| | | ggcauggaag ucacaccuuc gggaacgugg uugaccuaca caggugccau caaauuggau |
| | | gacaaagauc caaauuucaa agaucaaguc auuuugcuga auaagcauau ugacgcauac |
| | | aaaacauucc caccaacaga gccuaaaaag gacaaaaaga agaaggcuga ugaaacucaa |
| | | gccuuaccgc agagacagaa gaaacagcaa acugugacuc uucuuccugc ugcagauuug |
| | | gaugauuucu ccaaacaauu gcaacaaucc augagcagug cugacucaac ucaggccua |
| 16 | N Protein | MSDNGPQNQR NAPRITFGGP SDSTGSNQNG ERSGARSKQR RPQGLPNNTA SWFTALTQHG |
| | | KEDLKFPRGQ GVPINTNSSP DDQIGYYRRA TRRIRGGDGK MKDLSPRWYF YYLGTGPEAG |
| | | LPYGANKDGI IWVATEGALN TPKDHIGTRN PANNAAIVLQ LPQGTTLPKG FYAEGSRGGS |
| | | QASSRSSSRS RNSSRNSTPG SSRGTSPARM AGNGGDAALA LLLLDRLNQL ESKMSGKGQQ |
| | | QQGQTVTKKS AAEASKKPRQ KRTATKAYNV TQAFGRRGPE QTQGNFGDQE LIRQGTDYKH |
| | | WPQIAQFAPS ASAFFGMSRI GMEVTPSGTW LTYTGAIKLD DKDPNFKDQV ILLNKHIDAY |
| | | KTFPPTEPKK DKKKKADETQ ALPQRQKKQQ TVTLLPAADL DDFSKQLQQS MSSADSTQA |
| 17 | ORF3a from | atggatttgt ttatgagaat cttcacaatt ggaactgtaa ctttgaagca aggtgaaatc |
| | SARS-CoV-2 | aaggatgcta ctccttcaga ttttgttcgc gctactgcaa cgataccgat acaagcctca |
| | | ctccctttcg gatggcttat tgttggcgtt gcacttcttg ctgtttttca gagcgcttcc |
| | | aaaatcataa ccctcaaaaa gagatggcaa ctagcactct ccaagggtgt tcactttgtt |
| | | tgcaacttgc tgttgttgtt tgtaacagtt tactcacacc ttttgctcgt tgctgctggc |
| | | cttgaagccc cttttctcta tctttatgct ttagtctact tcttgcagag tataaacttt |
| | | gtaagaataa taatgaggct ttggctttgc tggaaatgcc gttccaaaaa cccattactt |
| | | tatgatgcca actattttct ttgctggcat actaattgtt acgactattg tataccttac |
| | | aatagtgtaa cttcttcaat tgtcattact tcaggtgatg gcacaacaag tcctatttct |
| | | gaacatgact accagattgg tggttatact gaaaaatggg aatctggagt aaaagactgt |
| | | gttgtattac acagttactt cacttcagac tattaccagc tgtactcaac tcaattgagt |
| | | acagacactg gtgttgaaca tgttaccttc ttcatctaca ataaaattgt tgatgagcct |
| | | gaagaacatg tccaaattca cacaatcgac ggttcatccg gagttgttaa tccagtaatg |
| | | gaaccaattt atgatgaacc gacgacgact actagcgtgc ctttgtaa |
| 18 | ORF3a from | auggauuugu uuaugagaau cuucacaauu ggaacuguaa cuuugaagca aggugaaauc |
| | SARS-CoV-2 | aaggaugcua cuccuucaga uuuuguucgc gcuacugcaa cgauaccgau acaagccuca |
| | | cucccuuucg gauggcuuau uguuggcguu gcacuucuug cuguuuuuca gagcgcuucc |
| | | aaaaucauaa cccucaaaaa gagauggcaa cuagcacucu ccaagggugu ucacuuuguu |
| | | ugcaacuugc uguuguuguu uguaacaguu uacucacacc uuuugcucgu ugcugcuggc |
| | | cuugaagccc cuuuucucua ucuuuaugcu uuagucuacu ucuugcagag uauaaacuuu |
| | | guaagaauaa uaaugaggcu uuggcuuugc uggaaaugcc guuccaaaaa cccauuccuu |
| | | uaugaugcca acuauuuucu uugcuggcau acuaauuguu acgacuauug uauaccuuac |
| | | aauaguguaa cuucuucaau ugucauuacu ucaggugaug gcacaacaag uccuauuucu |
| | | gaacaugacu accagauugg ugguuauacu gaaaaauggg aaucuggagu aaaagacugu |
| | | guuguauuac acaguuacuu cacuucagac uauuaccagc uguacucaac ucaauugagu |
| | | acagacacug guguugaaca uguuaccuuc uucaucuaca auaaaauugu ugaugagccu |
| | | gaagaacaug uccaaauuca cacaaucgac gguucauccg gaguuguuaa uccaguaaug |
| | | gaaccaauuu augaugaacc gacgacgacu acuagcgugc cuuuguaa |
| 19 | ORF3a | MDLFMRIFTI GTVTLKQGEI KDATPSDFVR ATATIPIQAS LPFGWLIVGV ALLAVFQSAS |
| | | KIITLKKRWQ LALSKGVHFV CNLLLLFVTV YSHLLLVAAG LEAPFLYLYA LVYFLQSINF |
| | | VRIIMRLWLC WKCRSKNPLL YDANYFLCWH TNCYDYCIPY NSVTSSIVIT SGDGTTSPIS |
| | | EHDYQIGGYT EKWESGVKDC VVLHSYFTSD YYQLYSTQLS TDTGVEHVTF FIYNKIVDEP |
| | | EEHVQIHTID GSSGVVNPVM EPIYDEPTTT TSVPL |
| 20 | E protein from | atgtactcat tcgtttcgga agagacaggt acgttaatag ttaatagcgt acttcttttt |
| | SARS-CoV-2 | cttgctttcg tggtattctt gctagttaca ctagccatcc ttactgcgct tcgattgtgt |
| | | gcgtactgct gcaatattgt taacgtgagt cttgtaaaac cttcttttta cgtttactct |
| | | cgtgttaaaa atctgaattc ttctagagtt cctgatcttc tggtctaa |
| 21 | E protein from | auguacucau ucguuucgga agagacaggu acguuaauag uuaauagcgu acuucuuuuu |
| | SARS-CoV-2 | cuugcuuucg ugguauucuu gcuaguuaca cuagccaucc uuacugcgcu ucgauugugu |
| | | gcguacugcu gcaauauugu uaacgugagu cuuguaaaac cuucuuuuua cguuuacucu |
| | | cguguuaaaa aucugaauuc uucuagaguu ccugaucuuc uggucuaa |
| 22 | E Protein | MYSFVSEETG TLIVNSVLLF LAFVVFLLVT LAILTALRLC AYCCNIVNVS LVKPSFYVYS |
| | | RVKNLNSSRV PDLLV |
| 23 | ORF3a + E | atggattt |
| | protein | gtttatgaga atcttcacaa ttggaactgt aactttgaag caaggtgaaa tcaaggatgc |
| | | tactccttca gattttgttc gcgctactgc aacgataccg atacaagcct cactcccttt |
| | | cggatggctt attgttggcg ttgcacttct tgctgttttt cagagcgctt ccaaaatcat |
| | | aaccctcaaa aagagatggc aactagcact ctccaagggt gttcactttg tttgcaactt |
| | | gctgttgttg tttgtaacag tttactcaca ccttttgctc gttgctgctg gccttgaagc |
| | | cccttttctc tatctttatg ctttagtcta cttcttgcag agtataaact ttgtaagaat |
| | | aataatgagg ctttggcttt gctggaaatg ccgttccaaa aacccattac tttatgatgc |
| | | caactatttt ctttgctggc atactaattg ttacgactat tgtatacctt acaatagtgt |
| | | aacttcttca attgtcatta cttcaggtga tggcacaaca agtcctattt ctgaacatga |
| | | ctaccagatt ggtggttata ctgaaaaatg ggaatctgga gtaaaagact gtgttgtatt |
| | | acacagttac ttcacttcag actattacca gctgtactca actcaattga gtacagacac |
| | | tggtgttgaa catgttacct tcttcatcta caataaaatt gttgatgagc ctgaagaaca |
| | | tgtccaaatt cacacaatcg acggttcatc cggagttgtt aatccagtaa tggaaccaat |
| | | ttatgatgaa ccgacgacga ctactagcgt gcctttgtaa gcacaagctg atgagtacga |
| | | acttatgtac tcattcgttt cggaagagac aggtacgtta atagttaata gcgtacttct |
| | | ttttcttgct ttcgtggtat tcttgctagt tacactagcc atccttactg cgcttcgatt |
| | | gtgtgcgtac tgctgcaata ttgttaacgt gagtcttgta aaaccttctt tttacgttta |
| | | ctctcgtgtt aaaaatctga attcttctag agttcctgat cttctggtct aaac |
| 24 | ORF3a + E | auggauuu |
| | protein | guuuaugaga aucuucacaa uuggaacugu aacuuugaag caaggugaaa ucaaggaugc |
| | | uacuccuuca gauuuuguuc gcgcuacugc aacgauaccg auacaagccu cacucccuuu |
| | | cggauggcuu auuguuggcg uugcacuucu ugcuguuuuu cagagcgcuu ccaaaaucau |
| | | aacccucaaa aagagauggc aacuagcacu cuccaagggu guucacuuug uuugcaacuu |
| | | gcuguuguug uuuguaacag uuuacucaca ccuuuugcuc guugcugcug gccuugaagc |
| | | cccuuuucuc uaucuuuaug cuuuagucua cuucuugcag aguauaaacu uuguaagaau |
| | | aauaaugagg cuuuggcuuu gcuggaaagu ccguuccaaa aacccauuac uuuaugaugc |
| | | caacuauuuu cuuugcuggc auacuaauug uuacgacuau uguauaccuu acaauagugu |
| | | aacuucuuca auugucauua cuucagguga uggcacaaca aguccuauuu cugaacauga |
| | | cuaccagauu ggugguuaua cugaaaaaug ggaaucugga guaaaagacu guguuguauu |
| | | acacaguuac uucacuucag acuauuacca gcuguacuca acucaauuga guacagacac |
| | | ugguguugaa cauguuaccu ucuucaucua caauaaaauu guugaugagc cugaagaaca |
| | | uguccaaauu cacacaaucg acgguucauc cggaguuguu aauccaguaa uggaaccaau |
| | | uuaugaugaa ccgacgacga cuacuagcgu gccuuuguaa gcacaagcug augaguacga |
| | | acuuauguac ucauucguuu cggaagagac agguacguua auaguuaaua gcguacuucu |
| | | uuuucuugcu uucgugguau ucuugcuagu uacacuagcc auccuuacug cgcuucgauu |
| | | gugugcguac ugcugcaaua uuguuaacgu gagucuugua aaaccuucuu uuuacguuua |
| | | cucucguguu aaaaaucuga auucuucuag aguuccugau cuucuggucu aaac |
| 25 | Intron between | actcatg cagaccacac aaggcag |
| | N Protein and | |
| | ORF 10 | |
| 26 | Intron between | acucaug cagaccacac aaggcag |
| | N Protein and | |
| | ORF 10 | |
| 27 | 5′UTR + | Nttaaaggtt tataccttcc caggtaacaa accaaccaac tttcgatctc ttgtagatct |
| | 1-208 of nsp1 + | gttctctaaa cgaactttaa aatctgtgtg gctgtcactc ggctgcatgc ttagtgcact |
| | 20-117 of | cacgcagtat aattaataac taattactgt cgttgacagg acacgagtaa ctcgtctatc |
| | ORF10 + 3′UTR | ttctgcaggc tgcttacggt ttcgtccgtg ttgcagccga tcatcagcac atctaggttt |
| | | cgtccgggtg tgaccgaaag gtaagatgga gagccttgtc cctggtttca acgagaaaac |
| | | acacgtccaa ctcagtttgc ctgttttaca ggttcgcgac gtgctcgtac gtggctttgg |
| | | agactccgtg gaggaggtct tatcagaggc acgtcaacat cttaaagatg gcacttgtgg |
| | | cttagtagaa gttgaaaaag gcgttttgcc tcaacttcga acagccctat gtgtcgcttt |
| | | tccgtttacg atatatagtc tactcttgtg cagaatgaat tctcgtaact acatagcaca |
| | | agtagatgta gttaacttta atctcacata gcaatcttta atcagtgtgt aacattaggg |
| | | aggacttgaa agagccacca cattttcacc gaggccacgc ggagtacgat cgagtgtaca |
| | | gtgaacaatg ctagggagag ctgcctatat ggaagagccc taatgtgtaa aattaatttt |
| | | agtagtgcta tccccatgtg attttaatag cttcttagga gaatgacaaa aaaaaaaaaa |
| | | aaaaaaaaaa aaaaaaaaaa |
| 28 | 5′UTR + | Nuuaaagguu uauaccuucc cagguaacaa accaaccaac uuucgaucuc uuguagaucu |
| | 1-208 of nsp1 + | guucucuaaa cgaacuuuaa aaucugugug gcugucacuc ggcugcaugc uuagugcacu |
| | 20-117 of | cacgcaguau aauuaauaac uaauuacugu cguugacagg acacgaguaa cucgucuauc |
| | ORF10 + 3′UTR | uucugcaggc ugcuuacggu uucguccgug uugcagccga ucaucagcac aucuagguuu |
| | | cguccgggug ugaccgaaag guaagaugga gagccuuguc ccugguuuca acgagaaaac |
| | | acacguccaa cucaguuugc cuguuuuaca gguucgcgac gugcucguac guggcuuugg |
| | | agacuccgug gaggaggucu uaucagaggc acgucaacau cuuaaagaug gcacuugugg |
| | | cuuaguagaa guugaaaaag gcguuuugcc ucaacuucga acagcccuau gugucgcuuu |
| | | uccguuuacg auauauaguc uacucuugug cagaaugaau ucucguaacu acauagcaca |
| | | aguagaugua guuaacuuua aucucacaua gcaaucuuua aucagugugu aacauuaggg |
| | | aggacuugaa agagccacca cauuuucacc gaggccacgc ggaguacgau cgaguguaca |
| | | gugaacaaug cuagggagag cugccuauau ggaagagccc uaauguguaa aauuaauuuu |
| | | aguagugcua uccccaugug auuuuaauag cuucuuagga gaaugacaaa aaaaaaaaaa |
| | | aaaaaaaaaa aaaaaaaaa |
| 29 | 5′UTR + | Nttaaaggtt tataccttcc caggtaacaa accaaccaac tttcgatctc ttgtagatct |
| | 1-207 of nsp1 + | gttctctaaa cgaactttaa aatctgtgtg gctgtcactc ggctgcatgc ttagtgcact |
| | 760-1015 of | cacgcagtat aattaataac taattactgt cgttgacagg acacgagtaa ctcgtctatc |
| | ORF3a + E | ttctgcaggc tgcttacggt ttcgtccgtg ttgcagccga tcatcagcac atctaggttt |
| | protein + | cgtccgggtg tgaccgaaag gtaagatgga gagccttgtc cctggtttca acgagaaaac |
| | 20-117 of | acacgtccaa ctcagtttgc ctgttttaca ggttcgcgac gtgctcgtac gtggctttgg |
| | ORF10 + 3′UTR | agactccgtg gaggaggtct tatcagaggc acgtcaacat cttaaagatg gcacttgtgg |
| | | cttagtagaa gttgaaaaag gcgttttgcc tcaacttcga acagccctat gtgcgaagga |
| | | gttgttaatc cagtaatgga accaatttat gatgaaccga cgacgactac tagcgtgcct |
| | | ttgtaagcac aagctgatga gtacgaactt atgtactcat tcgtttcgga agagacaggt |
| | | acgttaatag ttaatagcgt acttcttttt cttgctttcg tggtattctt gctagttaca |
| | | ctagccatcc ttactgcgct tcgattgtgt gcgtactgct gcaatattgt taacgtgagt |
| | | cttgtaaaac cttttcgctt ttccgtttac gatatatagt ctactcttgt gcagaatgaa |
| | | ttctcgtaac tacatagcac aagtagatgt agttaacttt aatctcacat agcaatcttt |
| | | aatcagtgtg taacattagg gaggacttga aagagccacc acattttcac cgaggccacg |
| | | cggagtacga tcgagtgtac agtgaacaat gctagggaga gctgcctata tggaagaggc |
| | | ctaatgtgta aaattaattt tagtagtgct atccccatgt gattttaata gcttcttagg |
| | | agaatgacaa aaaaaaaaaa aaaaaaaaaa aaaaaaaaaa a |
| | | *underlined nucleotides are optional |
| 30 | 5′UTR + | Nuuaaagguu uauaccuucc cagguaacaa accaaccaac uuucgaucuc uuguagaucu |
| | 1-207 of nsp1 + | guucucuaaa cgaacuuuaa aaucugugug gcugucacuc ggcugcaugc uuagugcacu |
| | 760-1015 of | cacgcaguau aauuaauaac uaauuacugu cguugacagg acacgaguaa cucgucuauc |
| | ORF3a + E | uucugcaggc ugcuuacggu uucguccgug uugcagccga ucaucagcac aucuagguuu |
| | protein + | cguccgggug ugaccgaaag guaagaugga gagccuuguc ccugguuuca acgagaaaac |
| | 20-117 of | acacguccaa cucaguuugc cuguuuuaca gguucgcgac gugcucguac guggcuuugg |
| | ORF10 + 3′UTR | agacuccgug gaggaggucu uaucagaggc acgucaacau cuuaaagaug gcacuugugg |
| | | cuuaguagaa guugaaaaag gcguuuugcc ucaacuucga acagcccuau gugcgaagga |
| | | guuguuaauc caguaaugga accaauuuau gaugaaccga cgacgacuac uagcgugccu |
| | | uuguaagcac aagcugauga guacgaacuu auguacucau ucguuucgga agagacaggu |
| | | acguuaauag uuaauagcgu acuucuuuuu cuugcuuucg ugguauucuu gcuaguuaca |
| | | cuagccaucc uuacugcgcu ucgauugugu gcguacugcu gcaauauugu uaacgugagu |
| | | cuuguaaaac cuuuucgcuu uuccguuuac gauauauagu cuacucuugu gcagaaugaa |
| | | uucucguaac uacauagcac aaguagaugu aguuaacuuu aaucucacau agcaaucuuu |
| | | aaucagugug uaacauuagg gaggacuuga aagagccacc acauuuucac cgaggccacg |
| | | cggaguacga ucgaguguac agugaacaau gcuagggaga gcugccuaua uggaagagcc |
| | | cuaaugugua aaauuaauuu uaguagugca auccccaugu gauuuuaaua gcuucuuagg |
| | | agaaugacaa aaaaaaaaaa aaaaaaaaaa aaaaaaaaaa a |
| | | *underlined nucleotides are optional |
| 31 | 5′UTR + | Nttaaaggtt tataccttcc caggtaacaa accaaccaac tttcgatctc ttgtagatct |
| | 1-207 of nsp1 + | gttctctaaa cgaactttaa aatctgtgtg gctgtcactc ggctgcatgc ttagtgcact |
| | 54-720 nsp15 + | cacgcagtat aattaataac taattactgt cgttgacagg acacgagtaa ctcgtctatc |
| | 205-1259 of N | ttctgcaggc tgcttacggt ttcgtccgtg ttgcagccga tcatcagcac atctaggttt |
| | protein + | cgtccgggtg tgaccgaaag gtaagatgga gagccttgtc cctggtttca acgagaaaac |
| | 20-117 of | acacgtccaa ctcagtttgc ctgttttaca ggttcgcgac gtgctcgtac gtggctttgg |
| | ORF10 + 3′UTR | agactccgtg gaggaggtct tatcagaggc acgtcaacat cttaaagatg gcacttgtgg |
| | | cttagtagaa gttgaaaaag gcgttttgcc tcaacttcga acagccctat gtgacagggt |
| | | gaagtaccag tttctatcat taataacact gtttacacaa aagttgatgg tgttgatgta |
| | | gaattgtttg aaaataaaac aacattacct gttaatgtag catttgagct ttgggctaag |
| | | cgcaacatta aaccagtacc agaggtgaaa atactcaata atttgggtgt ggacattgct |
| | | gctaatactg tgatctggga ctacaaaaga gatgctccag cacatatatc tactattggt |
| | | gtttgttcta tgactgacat agccaagaaa ccaactgaaa cgatttgtgc accactcact |
| | | gtcttttttg atggtagagt tgatggtcaa gtagacttat ttagaaatgc ccgtaatggt |
| | | gttcttatta cagaaggtag tgttaaaggt ttacaaccat ctgtaggtcc caaacaagct |
| | | agtcttaatg gagtcacatt aattggagaa gccgtaaaaa cacagttcaa ttattataag |
| | | aaagttgatg gtgttgtcca acaattacct gaaacttact ttactcagag tagaaattta |
| | | caagaattta aacccaggag tcaaatggaa attgatttct tagaattagc tatggatgaa |
| | | ttcattgaac ggtataaatt agaaggctat gccttcgaac atatcgttta tggagatttt |
| | | ggacaaggcg ttccaattaa caccaatagc agtccagatg accaaattgg ctactaccga |
| | | agagctacca gacgaattcg tggtggtgac ggtaaaatga aagatctcag tccaagatgg |
| | | tatttctact acctaggaac tgggccagaa gctggacttc cctatggtgc taacaaagac |
| | | ggcatcatat gggttgcaac tgagggagcc ttgaatacac caaaagatca cattggcacc |
| | | cgcaatcctg ctaacaatgc tgcaatcgtg ctacaacttc ctcaaggaac aacattgcca |
| | | aaaggcttct acgcagaagg gagcagaggc ggcagtcaag cctcttctcg ttcctcatca |
| | | cgtagtcgca acagttcaag aaattcaact ccaggcagca gtaggggaac ttctcctgct |
| | | agaatggctg gcaatggcgg tgatgctgct cttgctttgc tgctgcttga cagattgaac |
| | | cagcttgaga gcaaaatgtc tggtaaaggc caacaacaac aaggccaaac tgtcactaag |
| | | aaatctgctg ctgaggcttc taagaagcct cggcaaaaac gtactgccac taaagcatac |
| | | aatgtaacac aagctttcgg cagacgtggt ccagaacaaa cccaaggaaa ttttggggac |
| | | caggaactaa tcagacaagg aactgattac aaacattggc cgcaaattgc acaatttgcc |
| | | cccagcgctt cagcgttctt cggaatgtcg cgcattggca tggaagtcac accttcggga |
| | | acgtggttga cctacacagg tgccatcaaa ttggatgaca aagatccaaa tttcaaagat |
| | | caagtcattt tgctgaataa gcatattgac gcatacaaaa cattcccacc aacagagcct |
| | | aaaaaggaca aaaagaagaa ggctgatgaa actcaagcct taccgcagag acagaagaaa |
| | | cagcaaactg tgactcttct tcctgctgca gatttggatg atttctccaa acaattgcaa |
| | | caatccatga gcagtgctga ctcaactcag gcctatcgct tttccgttta cgatatatag |
| | | tctactcttg tgcagaatga attctcgtaa ctacatagca caagtagatg tagttaactt |
| | | taatctcaca tagcaatctt taatcagtgt gtaacattag ggaggacttg aaagagccac |
| | | cacattttca ccgaggccac gcggagtacg atcgagtgta cagtgaacaa tgctagggag |
| | | agctgcctat atggaagagc cctaatgtgt aaaattaatt ttagtagtgc tatccccatg |
| | | tgattttaat agcttcttag gagaatgaca aaaaaaaaaa aaaaaaaaaa aaaaaaaaaa |
| | | aa |
| 32 | 5′UTR + | Nuuaaagguu uauaccuucc cagguaacaa accaaccaac uuucgaucuc uuguagaucu |
| | 1-207 of nsp1 + | guucucuaaa cgaacuuuaa aaucugugug gcugucacuc ggcugcaugc uuagugcacu |
| | 54-720 nsp15 + | cacgcaguau aauuaauaac uaauuacugu cguugacagg acacgaguaa cucgucuauc |
| | 205-1259 of N | uucugcaggc ugcuuacggu uucguccgug uugcagccga ucaucagcac aucuagguuu |
| | protein + | cguccgggug ugaccgaaag guaagaugga gagccuuguc ccugguuuca acgagaaaac |
| | 20-117 of | acacguccaa cucaguuugc cuguuuuaca gguucgcgac gugcucguac guggcuuugg |
| | ORF10 + 3′UTR | agacuccgug gaggaggucu uaucagaggc acgucaacau cuuaaagaug gcacuugugg |
| | | cuuaguagaa guugaaaaag gcguuuugcc ucaacuucga acagcccuau gugacagggu |
| | | gaaguaccag uuucuaucau uaauaacacu guuuacacaa aaguugaugg uguugaugua |
| | | gaauuguuug aaaauaaaac aacauuaccu guuaauguag cauuugagcu uugggcuaag |
| | | cgcaacauua aaccaguacc agaggugaaa auacucaaua auuugggugu ggacauugcu |
| | | gcuaauacug ugaucuggga cuacaaaaga gaugcuccag cacauauauc uacuauuggu |
| | | guuuguucua ugacugacau agccaagaaa ccaacugaaa cgauuugugc accacucacu |
| | | gucuuuuuug augguagagu ugauggucaa guagacuuau uuagaaaugc ccguaauggu |
| | | guucuuauua cagaagguag uguuaaaggu uuacaaccau cuguaggucc caaacaagcu |
| | | agucuuaaug gagucacauu aauuggagaa gccguaaaaa cacaguucaa uuauuauaag |
| | | aaaguugaug guguugucca acaauuaccu gaaacuuacu uuacucagag uagaaauuua |
| | | caagaauuua aacccaggag ucaaauggaa auugauuucu uagaauuagc uauggaugaa |
| | | uucauugaac gguauaaauu agaaggcuau gccuucgaac auaucguuua uggagauuuu |
| | | ggacaaggcg uuccaauuaa caccaauagc aguccagaug accaaauugg cuacuaccga |
| | | agagcuacca gacgaauucg ugguggugac gguaaaauga aagaucucag uccaagaugg |
| | | uauuucuacu accuaggaac ugggccagaa gcuggacuuc ccuauggugc uaacaaagac |
| | | ggcaucauau ggguugcaac ugagggagcc uugaauacac caaaagauca cauuggcacc |
| | | cgcaauccug cuaacaaugc ugcaaucgug cuacaacuuc cucaaggaac aacauugcca |
| | | aaaggcuucu acgcagaagg gagcagaggc ggcagucaag ccucuucucg uuccucauca |
| | | cguagucgca acaguucaag aaauucaacu ccaggcagca guaggggaac uucuccugcu |
| | | agaauggcug gcaauggcgg ugaugcugcu cuugcuuugc ugcugcuuga cagauugaac |
| | | cagcuugaga gcaaaauguc ugguaaaggc caacaacaac aaggccaaac ugucacuaag |
| | | aaaucugcug cugaggcuuc uaagaagccu cggcaaaaac guacugccac uaaagcauac |
| | | aauguaacac aagcuuucgg cagacguggu ccagaacaaa cccaaggaaa uuuuggggac |
| | | caggaacuaa ucagacaagg aacugauuac aaacauuggc cgcaaauugc acaauuugcc |
| | | cccagcgcuu cagcguucuu cggaaugucg cgcauuggca uggaagucac accuucggga |
| | | acgugguuga ccuacacagg ugccaucaaa uuggaugaca aagauccaaa uuucaaagau |
| | | caagucauuu ugcugaauaa gcauauugac gcauacaaaa cauucccacc aacagagccu |
| | | aaaaaggaca aaaagaagaa ggcugaugaa acucaagccu uaccgcagag acagaagaaa |
| | | cagcaaacug ugacucuucu uccugcugca gauuuggaug auuucuccaa acaauugcaa |
| | | caauccauga gcagugcuga cucaacucag gccuaucgcu uuuccguuua cgauauauag |
| | | ucuacucuug ugcagaauga auucucguaa cuacauagca caaguagaug uaguuaacuu |
| | | uaaucucaca uagcaaucuu uaaucagugu guaacauuag ggaggacuug aaagagccac |
| | | cacauuuuca ccgaggccac gcggaguacg aucgagugua cagugaacaa ugcuagggag |
| | | agcugccuau auggaagagc ccuaaugugu aaaauuaauu uuaguagugc uauccccaug |
| | | ugauuuuaau agcuucuuag gagaaugaca aaaaaaaaaa aaaaaaaaaa aaaaaaaaaa |
| | | aa |
| 33 | 5′UTR + | Nttaaaggtt tataccttcc caggtaacaa accaaccaac tttcgatctc ttgtagatct |
| | 1-207 of nsp1 + | gttctctaaa cgaactttaa aatctgtgtg gctgtcactc ggctgcatgc ttagtgcact |
| | 54-720 nsp15 + | cacgcagtat aattaataac taattactgt cgttgacagg acacgagtaa ctcgtctatc |
| | 62-1016 of | ttctgcaggc tgcttacggt ttcgtccgtg ttgcagccga tcatcagcac atctaggttt |
| | ORF3a + E | cgtccgggtg tgaccgaaag gtaagatgga gagccttgtc cctggtttca acgagaaaac |
| | protein + | acacgtccaa ctcagtttgc ctgttttaca ggttcgcgac gtgctcgtac gtggctttgg |
| | 205-1259 of N | agactccgtg gaggaggtct tatcagaggc acgtcaacat cttaaagatg gcacttgtgg |
| | protein + | cttagtagaa gttgaaaaag gcgttttgcc tcaacttcga acagccctat gtgacagggt |
| | 20-117 of | gaagtaccag tttctatcat taataacact gtttacacaa aagttgatgg tgttgatgta |
| | ORF10 + 3′UTR | gaattgtttg aaaataaaac aacattacct gttaatgtag catttgagct ttgggctaag |
| | | cgcaacatta aaccagtacc agaggtgaaa atactcaata atttgggtgt ggacattgct |
| | | gctaatactg tgatctggga ctacaaaaga gatgctccag cacatatatc tactattggt |
| | | gtttgttcta tgactgacat agccaagaaa ccaactgaaa cgatttgtgc accactcact |
| | | gtcttttttg atggtagagt tgatggtcaa gtagacttat ttagaaatgc ccgtaatggt |
| | | gttcttatta cagaaggtag tgttaaaggt ttacaaccat ctgtaggtcc caaacaagct |
| | | agtcttaatg gagtcacatt aattggagaa gccgtaaaaa cacagttcaa ttattataag |
| | | aaagttgatg gtgttgtcca acaattacct gaaacttact ttactcagag tagaaattta |
| | | caagaattta aacccaggag tcaaatggaa attgatttct tagaattagc tatggatgaa |
| | | ttcattgaac ggtataaatt agaaggctat gccttcgaac atatcgttta tggagatttt |
| | | cgaaggagtt gttaatccag taatggaacc aatttatgat gaaccgacga cgactactag |
| | | cgtgcctttg taagcacaag ctgatgagta cgaacttatg tactcattcg tttcggaaga |
| | | gacaggtacg ttaatagtta atagcgtact tctttttctt gctttcgtgg tattcttgct |
| | | agttacacta gccatcctta ctgcgcttcg attgtgtgcg tactgctgca atattgttaa |
| | | cgtgagtctt gtaaaacctt tggacaaggc gttccaatta acaccaatag cagtccagat |
| | | gaccaaattg gctactaccg aagagctacc agacgaattc gtggtggtga cggtaaaatg |
| | | aaagatctca gtccaagatg gtatttctac tacctaggaa ctgggccaga agctggactt |
| | | ccctatggtg ctaacaaaga cggcatcata tgggttgcaa ctgagggagc cttgaataca |
| | | ccaaaagatc acattggcac ccgcaatcct gctaacaatg ctgcaatcgt gctacaactt |
| | | cctcaaggaa caacattggc aaaaggcttc tacgcagaag ggagcagagg cggcagtcaa |
| | | gcctcttctc gttcctcatc acgtagtcgc aacagttcaa gaaattcaac tccaggcagc |
| | | agtaggggaa cttctcctgc tagaatggct ggcaatggcg gtgatgctgc tcttgctttg |
| | | ctgctgcttg acagattgaa ccagcttgag agcaaaatgt ctggtaaagg ccaacaacaa |
| | | caaggccaaa ctgtcactaa gaaatctgct gctgaggctt ctaagaagcc tcggcaaaaa |
| | | cgtactgcca ctaaagcata caatgtaaca caagctttcg gcagacgtgg tccagaacaa |
| | | acccaaggaa attttgggga ccaggaacta atcagacaag gaactgatta caaacattgg |
| | | ccgcaaattg cacaatttgc ccccagcgct tcagcgttct tcggaatgtc gcgcattggc |
| | | atggaagtca caccttcggg aacgtggttg acctacacag gtgccatcaa attggatgac |
| | | aaagatccaa atttcaaaga tcaagtcatt ttgctgaata agcatattga cgcatacaaa |
| | | acattcccac caacagagcc taaaaaggac aaaaagaaga aggctgatga aactcaagcc |
| | | ttaccgcaga gacagaagaa acagcaaact gtgactcttc ttcctgctgc agatttggat |
| | | gatttctcca aacaattgca acaatccatg agcagtgctg actcaactca ggcctaaact |
| | | catgcagacc acacaaggca gtcgcttttc cgtttacgat atatagtcta ctcttgtgca |
| | | gaatgaattc tcgtaactac atagcacaag tagatgtagt taactttaat ctcacatagc |
| | | aatctttaat cagtgtgtaa cattagggag gacttgaaag agccaccaca ttttcaccga |
| | | ggccacgcgg agtacgatcg agtgtacagt gaacaatgct agggagagct gcctatatgg |
| | | aagagcccta atgtgtaaaa ttaattttag tagtgctatc cccatgtgat tttaatagct |
| | | tcttaggaga atgacaaaaa aaaaaaaaaa aaaaaaaaaa aaaaaaaa |
| | | *underlined nucleotides are optional |
| 34 | 5′UTR + | Nuuaaagguu uauaccuucc cagguaacaa accaaccaac uuucgaucuc uuguagaucu |
| | 1-207 of nsp1 + | guucucuaaa cgaacuuuaa aaucugugug gcugucacuc ggcugcaugc uuagugcacu |
| | 54-720 nsp15 + | cacgcaguau aauuaauaac uaauuacugu cguugacagg acacgaguaa cucgucuauc |
| | 62-1016 of | uucugcaggc ugcuuacggu uucguccgug uugcagccga ucaucagcac aucuagguuu |
| | ORF3a + E | cguccgggug ugaccgaaag guaagaugga gagccuuguc ccugguuuca acgagaaaac |
| | protein + | acacguccaa cucaguuugc cuguuuuaca gguucgcgac gugcucguac guggcuuugg |
| | 205-1259 of N | agacuccgug gaggaggucu uaucagaggc acgucaacau cuuaaagaug gcacuugugg |
| | protein + | cuuaguagaa guugaaaaag gcguuuugcc ucaacuucga acagcccuau gugacagggu |
| | 20-117 of | gaaguaccag uuucuaucau uaauaacacu guuuacacaa aaguugaugg uguugaugua |
| | ORF10 + 3′UTR | gaauuguuug aaaauaaaac aacauuaccu guuaauguag cauuugagcu uugggcuaag |
| | | cgcaacauua aaccaguacc agaggugaaa auacucaaua auuugggugu ggacauugcu |
| | | gcuaauacug ugaucuggga cuacaaaaga gaugcuccag cacauauauc uacuauuggu |
| | | guuuguucua ugacugacau agccaagaaa ccaacugaaa cgauuugugc accacucacu |
| | | gucuuuuuug augguagagu ugauggucaa guagacuuau uuagaaaugc ccguaauggu |
| | | guucuuauua cagaagguag uguuaaaggu uuacaaccau cuguaggucc caaacaagcu |
| | | agucuuaaug gagucacauu aauuggagaa gccguaaaaa cacaguucaa uuauuauaag |
| | | aaaguugaug guguugucca acaauuaccu gaaacuuacu uuacucagag uagaaauuua |
| | | caagaauuua aacccaggag ucaaauggaa auugauuucu uagaauuagc uauggaugaa |
| | | uucauugaac gguauaaauu agaaggcuau gccuucgaac auaucguuua uggagauuuu |
| | | cgaaggaguu guuaauccag uaauggaacc aauuuaugau gaaccgacga cgacuacuag |
| | | cgugccuuug uaagcacaag cugaugagua cgaacuuaug uacucauucg uuucggaaga |
| | | gacagguacg uuaauaguua auagcguacu ucuuuuucuu gcuuucgugg uauucuugcu |
| | | aguuacacua gccauccuua cugcgcuucg auugugugcg uacugcugca auauuguuaa |
| | | cgugagucuu guaaaaccuu uggacaaggc guuccaauua acaccaauag caguccagau |
| | | gaccaaauug gcuacuaccg aagagcuacc agacgaauuc guggugguga cgguaaaaug |
| | | aaagaucuca guccaagaug guauuucuac uaccuaggaa cugggccaga agcuggacuu |
| | | cccuauggug cuaacaaaga cggcaucaua uggguugcaa cugagggagc cuugaauaca |
| | | ccaaaagauc acauuggcac ccgcaauccu gcuaacaaug cugcaaucgu gcuacaacuu |
| | | ccucaaggaa caacauugcc aaaaggcuuc uacgcagaag ggagcagagg cggcagucaa |
| | | gccucuucuc guuccucauc acguagucgc aacaguucaa gaaauucaac uccaggcagc |
| | | aguaggggaa cuucuccugc uagaauggcu ggcaauggcg uggaugcugc ucuugcuuug |
| | | cugcugcuug acagauugaa ccagcuugag agcaaaaugu cugguaaagg ccaacaacaa |
| | | caaggccaaa cugucacuaa gaaaucugcu gcugaggcuu cuaagaagcc ucggcaaaaa |
| | | cguacugcca cuaaagcaua caauguaaca caagcuuucg gcagacgugg uccagaacaa |
| | | acccaaggaa auuuugggga ccaggaacua aucagacaag gaacugauua caaacauugg |
| | | ccgcaaauug cacaauuugc ccccagcgcu ucagcguucu ucggaauguc gcgcauuggc |
| | | auggaaguca caccuucggg aacgugguug accuacacag gugccaucaa auuggaugac |
| | | aaagauccaa auuucaaaga ucaagucauu uugcugaaua agcauauuga cgcauacaaa |
| | | acauucccac caacagagcc uaaaaaggac aaaaagaaga aggcugauga aacucaagcc |
| | | uuaccgcaga gacagaagaa acagcaaacu gugacucuuc uuccugcugc agauuuggau |
| | | gauuucucca aacaauugca acaauccaug agcagugcug acucaacuca ggccuaaacu |
| | | caugcagacc acacaaggca gucgcuuuuc cguuuacgau auauagucua cucuugugca |
| | | gaaugaauuc ucguaacuac auagcacaag uagauguagu uaacuuuaau cucacauagc |
| | | aaucuuuaau caguguguaa cauuagggag gacuugaaag agccaccaca uuuucaccga |
| | | ggccacgcgg aguacgaucg aguguacagu gaacaaugcu agggagagcu gccuauaugg |
| | | aagagcccua auguguaaaa uuaauuuuag uagugcuauc cccaugugag uuuaauagcu |
| | | ucuuaggaga augacaaaaa aaaaaaaaaa aaaaaaaaaa aaaaaaaa |
| | | *underlined nucleotides are optional |
| 35 | 5′UTR + | Nttaaaggtt tataccttcc caggtaacaa accaaccaac tttcgatctc ttgtagatct |
| | 1-524 of nsp1 + | gttctctaaa cgaactttaa aatctgtgtg gctgtcactc ggctgcatgc ttagtgcact |
| | 54-720 nsp15 + | cacgcagtat aattaataac taattactgt cgttgacagg acacgagtaa ctcgtctatc |
| | 62-1016 of | ttctgcaggc tgcttacggt ttcgtccgtg ttgcagccga tcatcagcac atctaggttt |
| | ORF3a + E | cgtccgggtg tgaccgaaag gtaagatgga gagccttgtc cctggtttca acgagaaaac |
| | protein + | acacgtccaa ctcagtttgc ctgttttaca ggttcgcgac gtgctcgtac gtggctttgg |
| | 205-1259 of N | agactccgtg gaggaggtct tatcagaggc acgtcaacat cttaaagatg gcacttgtgg |
| | protein + | cttagtagaa gttgaaaaag gcgttttgcc tcaacttcga acagccctat gtgtcatcaa |
| | ORF10 + 3′UTR | acgttcggat gctcgaactg cacctcatgg tcatgttatg gttgagctgg tagcagaact |
| | | cgaaggcatt cagtacggtc gtagtggtga gacacttggt gtccttgtcc ctcatgtggg |
| | | cgaaatacca gtggcttacc gcaaggttct tcttcgtaag aacggtaata aaggagctgg |
| | | tggccatagt tacggcgccg atctaaagtc atttgactta ggcgacgagc ttggcactga |
| | | tccttatgaa gattttcaag aaaactggaa cactaaacat agcagtggtg ttacccgtga |
| | | actcatgcga cagggtgaag taccagtttc tatcattaat aacactgttt acacaaaagt |
| | | tgatggtgtt gatgtagaat tgtttgaaaa taaaacaaca ttacctgtta atgtagcatt |
| | | tgagctttgg gctaagcgca acattaaacc agtaccagag gtgaaaatac tcaataattt |
| | | gggtgtggac attgctgcta atactgtgat ctgggactac aaaagagatg ctccagcaca |
| | | tatatctact attggtgttt gttctatgac tgacatagcc aagaaaccaa ctgaaacgat |
| | | ttgtgcacca ctcactgtct tttttgatgg tagagttgat ggtcaagtag acttatttag |
| | | aaatgcccgt aatggtgttc ttattacaga aggtagtgtt aaaggtttac aaccatctgt |
| | | aggtcccaaa caagctagtc ttaatggagt cacattaatt ggagaagccg taaaaacaca |
| | | gttcaattat tataagaaag ttgatggtgt tgtccaacaa ttacctgaaa cttactttac |
| | | tcagagtaga aatttacaag aatttaaacc caggagtcaa atggaaattg atttcttaga |
| | | attagctatg gatgaattca ttgaacggta taaattagaa ggctatgcct tcgaacatat |
| | | cgtttatgga gattttcgaa ggagttgtta atccagtaat ggaaccaatt tatgatgaac |
| | | cgacgacgac tactagcgtg cctttgtaag cacaagctga tgagtacgaa cttatgtact |
| | | cattcgtttc ggaagagaca ggtacgttaa tagttaatag cgtacttctt tttcttgctt |
| | | tcgtggtatt cttgctagtt acactagcca tccttactgc gcttcgattg tgtgcgtact |
| | | gctgcaatat tgttaacgtg agtcttgtaa aacctttgga caaggcgttc caattaacac |
| | | caatagcagt ccagatgacc aaattggcta ctaccgaaga gctaccagac gaattcgtgg |
| | | tggtgacggt aaaatgaaag atctcagtcc aagatggtat ttctactacc taggaactgg |
| | | gccagaagct ggacttccct atggtgctaa caaagacggc atcatatggg ttgcaactga |
| | | gggagccttg aatacaccaa aagatacact tggcacccgc aatcctgcta acaatgctgc |
| | | aatcgtgcta caacttcctc aaggaacaac attgccaaaa ggcttctacg cagaagggag |
| | | cagaggcggc agtcaagcct cttctcgttc ctcatcacgt agtcgcaaca gttcaagaaa |
| | | ttcaactcca ggcagcagta ggggaacttc tcctgctaga atggctggca atggcggtga |
| | | tgctgctctt gctttgctgc tgcttgacag attgaaccag cttgagagca aaatgtctgg |
| | | taaaggccaa caacaacaag gccaaactgt cactaagaaa tctgctgctg aggcttctaa |
| | | gaagcctcgg caaaaacgta ctgccactaa agcatacaat gtaacacaag ctttcggcag |
| | | acgtggtcca gaacaaaccc aaggaaattt tggggaccag gaactaatca gacaaggaac |
| | | tgattacaaa cattggccgc aaattgcaca atttgccccc agcgcttcag cgttcttcgg |
| | | aatgtcgcgc attggcatgg aagtcacacc ttcgggaacg tggttgacct acacaggtgc |
| | | catcaaattg gatgacaaag atccaaattt caaagatcaa gtcattttgc tgaataagca |
| | | tattgacgca tacaaaacat tcccaccaac agagcctaaa aaggacaaaa agaagaaggc |
| | | tgatgaaact caagccttac cgcagagaca gaagaaacag caaactgtga ctcttcttcc |
| | | tgctgcagat ttggatgatt tctccaaaca attgcaacaa tccatgagca gtgctgactc |
| | | aactcaggcc taaactcatg cagaccacac aaggcagatg ggctatataa acgttttcgc |
| | | ttttccgttt acgatatata gtctactctt gtgcagaatg aattctcgta actacatagc |
| | | acaagtagat gtagttaact ttaatctcac atagcaatct ttaatcagtg tgtaacatta |
| | | gggaggactt gaaagagcca ccacattttc accgaggcca cgcggagtac gatcgagtgt |
| | | acagtgaaca atgctaggga gagctgccta tatggaagag ccctaatgtg taaaattaat |
| | | tttagtagtg ctatccccat gtgattttaa tagcttctta ggagaatgac aaaaaaaaaa |
| | | aaaaaaaaaa aaaaaaaaaa aaa |
| | | *underlined nucleotides are optional |
| 36 | 5′UTR + | guucucuaaa cgaacuuuaa aaucugugug gcugucacuc ggcugcaugc uuagugcacu |
| | 1-524 of nsp1 + | cacgcaguau aauuaauaac uaauuacugu cguugacagg acacgaguaa cucgucuauc |
| | 54-720 nsp15 + | uucugcaggc ugcuuacggu uucguccgug uugcagccga ucaucagcac aucuagguuu |
| | 62-1016 of | cguccgggug ugaccgaaag guaagaugga gagccuuguc ccugguuuca acgagaaaac |
| | ORF3a + E | acacguccaa cucaguuugc cuguuuuaca gguucgcgac gugcucguac guggcuuugg |
| | protein + | agacuccgug gaggaggucu uaucagaggc acgucaacau cuuaaagaug gcacuugugg |
| | 205-1259 of N | cuuaguagaa guugaaaaag gcguuuugcc ucaacuucga acagcccuau gugucaucaa |
| | protein + | acguucggau gcucgaacug caccucaugg ucauguuaug guugagcugg uagcagaacu |
| | ORF10 + 3′UTR | cgaaggcauu caguacgguc guagugguga gacacuuggu guccuugucc cucauguggg |
| | | cgaaauacca guggcuuacc gcaagguucu ucuucguaag aacgguaaua aaggagcugg |
| | | uggccauagu uacggcgccg aucuaaaguc auuugacuua ggcgacgagc uuggcacuga |
| | | uccuuaugaa gauuuucaag aaaacuggaa cacuaaacau agcaguggug uuacccguga |
| | | acucaugcga cagggugaag uaccaguuuc uaucauuaau aacacuguuu acacaaaagu |
| | | ugaugguguu gauguagaau uguuugaaaa uaaaacaaca uuaccuguua auguagcauu |
| | | ugagcuuugg gcuaagcgca acauuaaacc aguaccagag gugaaaauac ucaauaauuu |
| | | ggguguggac auugcugcua auacugugau cugggacuac aaaagagaug cuccagcaca |
| | | uauaucuacu auugguguuu guucuaugac ugacauagcc aagaaaccaa cugaaacgau |
| | | uugugcacca cucacugucu uuuuugaugg uagaguugau ggucaaguag acuuauuuag |
| | | aaaugcccgu aaugguguuc uuauuacaga agguaguguu aaagguuuac aaccaucugu |
| | | aggucccaaa caagcuaguc uuaauggagu cacauuaauu ggagaagccg uaaaaacaca |
| | | guucaauuau uauaagaaag uugauggugu uguccaacaa uuaccugaaa cuuacuuuac |
| | | ucagaguaga aauuuacaag aauuuaaacc caggagucaa auggaaauug auuucuuaga |
| | | auuagcuaug gaugaauuca uugaacggua uaaauuagaa ggcuaugccu ucgaacauau |
| | | cguuuaugga gauuuucgaa ggaguuguua auccaguaau ggaaccaauu uaugaugaac |
| | | cgacgacgac uacuagcgug ccuuuguaag cacaagcuga ugaguacgaa cuuauguacu |
| | | cauucguuuc ggaagagaca gguacguuaa uaguuaauag cguacuucuu uuucuugcuu |
| | | ucgugguauu cuugcuaguu acacuagcca uccuuacugc gcuucgauug ugugcguacu |
| | | gcugcaauau uguuaacgug agucuuguaa aaccuuugga caaggcguuc caauuaacac |
| | | caauagcagu ccagaugacc aaauuggcua cuaccgaaga gcuaccagac gaauucgugg |
| | | uggugacggu aaaaugaaag aucucagucc aagaugguau uucuacuacc uaggaacugg |
| | | gccagaagcu ggacuucccu auggugcuaa caaagacggc aucauauggg uugcaacuga |
| | | gggagccuug aauacaccaa aagaucacau uggcacccgc aauccugcua acaaugcugc |
| | | aaucgugcua caacuuccuc aaggaacaac auugccaaaa ggcuucuacg cagaagggag |
| | | cagaggcggc agucaagccu cuucucguuc cucaucacgu agucgcaaca guucaagaaa |
| | | uucaacucca ggcagcagua ggggaacuuc uccugcuaga auggcuggca auggcgguga |
| | | ugcugcucuu gcuuugcugc ugcuugacag auugaaccag cuugagagca aaaugucugg |
| | | uaaaggccaa caacaacaag gccaaacugu cacuaagaaa ucugcugcug aggcuucuaa |
| | | gaagccucgg caaaaacgua cugccacuaa agcauacaau guaacacaag cuuucggcag |
| | | acguggucca gaacaaaccc aaggaaauuu uggggaccag gaacuaauca gacaaggaac |
| | | ugauuacaaa cauuggccgc aaauugcaca auuugccccc agcgcuucag cguucuucgg |
| | | aaugucgcgc auuggcaugg aagucacacc uucgggaacg ugguugaccu acacaggugc |
| | | caucaaauug gaugacaaag auccaaauuu caaagaucaa gucauuuugc ugaauaagca |
| | | uauugacgca uacaaaacau ucccaccaac agagccuaaa aaggacaaaa agaagaaggc |
| | | ugaugaaacu caagccuuac cgcagagaca gaagaaacag caaacuguga cucuucuucc |
| | | ugcugcagau uuggaugauu ucuccaaaca auugcaacaa uccaugagca gugcugacuc |
| | | aacucaggcc uaaacucaug cagaccacac aaggcagaug ggcuauauaa acguuuucgc |
| | | uuuuccguuu acgauauaua gucuacucuu gugcagaaug aauucucgua acuacauagc |
| | | acaaguagau guaguuaacu uuaaucucac auagcaaucu uuaaucagug uguaacauua |
| | | gggaggacuu gaaagagcca ccacauuuuc accgaggcca cgcggaguac gaucgagugu |
| | | acagugaaca augcuaggga gagcugccua uauggaagag cccuaaugug uaaaauuaau |
| | | uuuaguagug cuauccccau gugauuuuaa uagcuucuua ggagaaugac aaaaaaaaaa |
| | | aaaaaaaaaa aaaaaaaaaa aaa |
| | | *underlined nucleotides are optional |
| 37 | Primer 1 | cgcctacact aattcgtcct ga |
| 38 | Primer 2 | tgagtcatgg ctgtgattac g |
| 39 | Primer 3 | gacttaaaac actgagccgt ac |
| 40 | ORF3a-E | gcacaagctg atgagtacga actt |
| | Protein Intron | |
| 41 | ORF3a-E | gcacaagcug augaguacga acuu |
| | Protein Intron | |
| 42 | T7 Promoter | taatacgact cactatagg |
| 43 | Primer 4 (F) | agcttggca ctgatcctt atg |
| 44 | Primer 4 (B) | acatcaaca ccatcaacttt tgtg |
| 45 | Probe | FAM/ttacccgtga actcatgcga cagg/IBFQ |
| 46 | Primer 5 (F) | atcagaggca cgtcaacatc |
| 47 | Primer 5 (B) | ttcattctgc acaagagtag acg |
| 48 | Probe | FAM/agccctatgt gtcgcttttc cgt/IBFQ |
| 49 | Primer 6 (F) | ccctgtgggt tttacactta a |
| 50 | Primer 6 (B) | acgattgtgc atcagctga |
| 51 | Probe | FAM/ccgtctgcgg tatgtggaaa ggttatg/IBFQ-3 |
| 52 | Primer 7 (F) | aggattcata tgtgggcgat g |
| 53 | Primer 7 (B) | agctcattgt agaaggtgtg g |
| 54 | Probe | FAM/agcacggcat cgtcaccaac t/IBFQ |

1. A recombinant nucleic acid construct encoding a defective interfering coronaviridae virus-like particle, said recombinant construct comprising:
a nucleotide sequence encoding coronaviridae replication signals comprising:
a nucleotide sequence from the coronaviridae virus 5′ untranslated region (5′ UTR), and
a nucleotide sequence from the coronaviridae virus 3′ untranslated region (3′ UTR), wherein said 3′ UTR nucleotide sequence is positioned 3′ to the 5′ UTR nucleotide sequence, and wherein said nucleotide sequence of said recombinant nucleic acid construct does not encode one or more functional coronaviridae proteins

2. The recombinant nucleic acid construct of claim 1, wherein the coronaviridae virus is a human coronavirus.

3. The recombinant nucleic acid construct of claim 2, wherein the human coronavirus is severe acute respiratory syndrome coronavirus 2.

4. (canceled)

5. The recombinant nucleic acid construct of claim 1, wherein the construct comprises the entire 5′ UTR nucleotide sequence and the entire 3′ UTR nucleotide sequence of the coronaviridae virus.

6. The recombinant nucleic acid construct of claim 1, wherein the nucleotide sequence encoding the coronaviridae virus replication signals further comprises:
a nucleotide sequence encoding a portion of the coronaviridae virus non-structural protein 1 (nsp1), wherein said nsp1 encoding nucleotide sequence is positioned 3′ to the 5′ UTR nucleotide sequence of the construct.

7. The recombinant nucleic acid construct of claim 1, wherein the nucleotide sequence encoding the coronaviridae virus replication signals further comprises:
a nucleotide sequence encoding a portion of the coronaviridae virus ORF10, wherein said ORF10 encoding nucleotide sequence is positioned 5′ to the 3′ UTR nucleotide sequence of the construct.

8. The recombinant nucleic acid construct of claim 1, wherein the recombinant nucleic acid construct further comprises:
a nucleotide sequence encoding a coronaviridae virus packaging signal, wherein said packaging signal nucleotide sequence is positioned 3′ to the 5′ UTR nucleotide sequence of the construct.

9. The recombinant nucleic acid construct of claim 8, wherein the nucleotide sequence encoding the coronaviridae virus packaging signal comprises a nucleotide sequence encoding a portion of the coronaviridae virus non-structural protein 15 (nsp15).

10. The recombinant nucleic acid construct of claim 8, wherein the nucleotide sequence encoding the coronaviridae virus packaging signal comprises a nucleotide sequence encoding a portion of the coronaviridae virus N protein.

11. The recombinant nucleic acid construct of claim 1, wherein the recombinant nucleic acid construct further comprises:
a further nucleotide sequence encoding a portion of ORF3a, a portion of E protein, or a portion of ORF3a and E protein, wherein said further nucleotide sequence portion is positioned 5′ to the 3′ UTR nucleotide sequence.

12. The recombinant nucleic acid construct of claim 1, further comprising:
a promoter sequence operatively coupled to the 5′ UTR nucleotide sequence of the construct.

13. The recombinant nucleic acid construct of claim 1, wherein the nucleic acid construct comprises:
a nucleotide sequence selected from the group consisting of SEQ ID NO: 27, SEQ ID NO: 29, SEQ ID NO: 31, SEQ ID NO: 33 and SEQ ID NO: 35, or
a nucleotide sequence having at least 80% sequence identity to SEO ID NO: 27, SEO ID NO: 29, SEQ ID NO: 31, SEQ ID NO: 33 or SEQ ID NO: 35.

14.-18. (canceled)

19. The recombinant nucleic acid construct of claim 13, wherein the construct further comprises one or more primer/probe nucleotide sequences selected from the group consisting of SEQ ID NO: 37, SEQ ID NO: 38, and SEQ ID NO: 39.

20. (canceled)

21. The recombinant nucleic acid construct of claim 1, wherein the nucleic acid construct comprises:
a nucleotide sequence selected from the group consisting of SEQ ID NO: 28, SEQ ID NO: 30, SEO ID NO: 32, SEO ID NO: 34, and SEQ ID NO: 36, or
a nucleotide sequence having at least 80% sequence identity to SEQ ID NO: 28, SEQ ID NO: 30, SEQ ID NO: 32, SEQ ID NO: 34, or SEQ ID NO: 36.

22.-26. (canceled)

27. A vector comprising the recombinant nucleic acid construct of claim 1.

28. A host cell comprising the vector of claim 27.

29. The host cell of claim 28, wherein said host cell further comprises a helper virus.

30. A defective interfering coronaviridae virus-like particle produced from the recombinant nucleic acid construct of claim 1.

31.-32. (canceled)

33. A pharmaceutical composition comprising:
the defective interfering coronaviridae virus-like particle of claim 30, and a pharmaceutically acceptable carrier.

34. A method of treating a subject infected with a coronaviridae virus, said method comprising:
administering to said subject the pharmaceutical composition of claim 33 in an amount effective to impair replication and spread of the coronaviridae virus in the patient.

35.-37. (canceled)
